(19) United States
(12) Patent Application Publication
(10) Pub. No.: US 2024/0358889 A1
(43) Pub. Date: Oct. 31, 2024

(54) PEPTIDE-CONTAINING HYBRID HYDROGEL FOR BONE REGENERATION

(71) Applicant: Ramot at Tel-Aviv University Ltd., Tel-Aviv (IL)

(72) Inventors: Lihi ADLER-ABRAMOVICH, Tel Aviv (IL); Michal HALPERIN-STERNFELD, Tel Aviv (IL); Moran AVIV, Tel Aviv (IL); Itzhak BINDERMAN, Tel Aviv (IL)

(73) Assignee: Ramot at Tel-Aviv University Ltd., Tel-Aviv (IL)

(21) Appl. No.: 18/766,738

(22) Filed: Jul. 9, 2024

Related U.S. Application Data
(63) Continuation-in-part of application No. PCT/IL2023/050042, filed on Jan. 13, 2023.
(60) Provisional application No. 63/299,082, filed on Jan. 13, 2022.

(51) Int. Cl. A61L 27/20; A61L 27/22; A61L 27/52

(52) U.S. Cl. CPC A61L 27/20; A61L 27/227; A61L 27/52; A61L 2430/02

(57) ABSTRACT

Methods and compositions employing hybrid hydrogels formed of high molecular weight hyaluronic acid and a plurality of self-assembled peptides in inducing bone regeneration are provided.

### RELATED APPLICATIONS

This application is a Continuation of PCT Patent Application No. PCT/IL2023/050042 having International filing date of Jan. 13, 2023, which claims the benefit of priority under 35 USC § 119(e) of U.S. Provisional Patent Application No. 63/299,082 filed on Jan. 13, 2022. The contents of the above applications are all incorporated by reference as if fully set forth herein in their entirety.

### FIELD AND BACKGROUND OF THE INVENTION

The present invention, in some embodiments thereof, relates to therapy and. More particularly, but not exclusively, to compositions comprising hybrid hydrogels made of self-assembled peptide(s) and biocompatible polymer(s) for use in bone regeneration.

Reconstruction of bone defects that cannot heal spontaneously, i.e., critical-sized bone defects, continues to be an enormous clinical challenge. Such defects may result from trauma, inflammation, or following bone tumor resections. Although autogenic, allogeneic, and xenogeneic bone materials have been widely applied to treat such defects, they possess potential limitations (Stevens, 2008), including limited availability and donor site morbidity, potential immunogenicities, and risk for disease transmission.

The classic tissue engineering approach, involving the use of living cells, growth factors, and a basic scaffold, has emerged as a promising tool to address these issues (Vacanti and Langer, 1999). However, despite the undoubted potential held by this approach, it still involves ex-vivo cell manipulation, which may have immunogenic and tumorigenic potential. An alternative tissue engineering strategy that eliminates the need for exogenous cells is developing cell-free scaffolds that can utilize the recipient's endogenous cells for in situ tissue regeneration. Such scaffolds are designed to mimic the ability of the natural extracellular matrix (ECM) to effectively recruit host cells to the defect site while providing structural support for the recruited cells to promote angiogenesis and osteogenesis (Franz et al., 2011).

Hydrogels are of particular interest as cell-ingrowth scaffolds for tissue engineering and bone regeneration, as they offer a three-dimensional (3D) network for cell attachment and growth (Bai et al., 2018, Derkus et al., 2020, Cheng et al., 2020). Specifically, naturally occurring polysaccharides, including hyaluronic acid (HA), alginate (Alg), and chitosan have elicited an immense amount of attention due to their high biocompatibility, biodegradability, low immunogenicity, and abundance in nature (Witzler et al., 2019). Nevertheless, their usefulness has been limited by unsatisfactory mechanical properties and a high degradation rate (Stevens, 2008). To overcome these drawbacks, polysaccharides are often chemically cross-linked (Witzler et al., 2019).

Hyaluronic acid (HA) is an extracellular matrix component, present in all biological tissues and body fluids in various molecular sizes. HA biodegradation is mainly mediated by members of a family of enzymes called hyaluronidases (HYAL1 and HYAL2) through the CD44 receptor in macrophages. When an inflammatory response occurs, immune cells, including monocytes and macrophages, secrete inflammatory mediators such as nitric oxide (NO), tumor necrosis factor (TNF)-α, and interleukin (IL)-6. During inflammation, high molecular weight (e.g., of at least 1,000 kDa) HA undergoes a decrease in chain length, most likely owing to hyaluronidase activity or cleavage by reactive oxygen species (ROS) produced in the tissue. HA uptake and fragmentation by macrophages are thought to be important for resolution of inflammation.

Toll-like receptors (TLRs) in macrophages are critical components of the innate immune response, responding to a wide range of chemicals produced by bacteria, viruses, and fungi. TLR-4 can be directly stimulated by LPS and low-molecular-weight HA, leading to activation of the NF-κB pathway (Voelcker et al., Exp. Derm. 2008, Vol. 17(2), pp. 100-107). High MW HA has been shown to bind directly to TLR-4 receptor on macrophages, thus preventing LPS activation of macrophages. High MW HA was also reported to exert antiangiogenic, immunosuppressive, and anti-inflammatory effects, whereby HA having lower molecular weight was reported to exhibit pro-inflammatory, pro-angiogenic and immunostimulatory properties.

Hybrid composite hydrogels are reviewed, for example, in Jia and Kiick in Macromol Biosci. 2009 Feb. 11; 9(2): 140-156, and references cited therein. Several hybrid hydrogels made of hyaluronic acid and polymers such as PEG, chitosan, cellulose and alginate have been reported. Hybrid hydrogels made of hyaluronic acid and other polysaccharides and proteins such as collagen, gelatin and fibrin have also been reported. Hydrogel matrices made of hyaluronic acid derivatized by a cell adhesive peptide fragment are disclosed, for example, in U.S. Pat. Nos. 5,834,029 and 6,156,572. Hydrogels made of hyaluronic acid modified with Nodo-66 antagonist have been reported in Hou et al., J. Neurosci. Met. 137:519-529, 2005.

An alternative method to strengthen polysaccharides is by integrating them with self-assembling short peptide hydrogelators to create composite hydrogels with improved and tunable mechanical properties (Ghosh et al., 2019, Aviv et al., 2018, Gong et al., 2016, Halperin-Sternfeld et al., 2022, WO 2011/151832).

Self-supporting hydrogels are formed by self-assembly of short peptides modified with aromatic groups such as 9-fluorenylmethoxycarbonyl (Fmoc) (Debnath et al., 2019). One of the most studied short aromatic peptides is FmocFF, a dipeptide protected by an aromatic group that can self-assemble into fibrillar hydrogels (Tikhonova et al., 2021).

FmocFF has recently been used in combination with other materials such as polysaccharides, proteins, and peptides to form hybrid systems. Specifically, combining FmocFF with polysaccharides enabled the formation of stable hydrogels without the need for cross-linking agents (Huang et al., 2011, Aviv et al., 2018, Ghosh et al., 2019, Gong et al., 2016). Huang et al. fabricated a composite hydrogel from FmocFF and the polysaccharide, konjac glucomannan, for the sustained delivery of hydrophobic drugs (Huang et al., 2011). The composite hydrogel had a nanofibrous architecture and displayed high rigidity when compared to FmocFF alone (Huang et al., 2011). A similar improvement in the mechanical properties was observed when FmocFF was integrated with Alginate and HA to form composite hydrogels (Aviv et al., 2018, Ghosh et al., 2019).

However, improvement in mechanical properties may not be enough to induce regeneration of high-quality dense, vascularized and functional bone.

Additional background art includes WO 2011/151832, WO2011/151832, WO 2007/0403048, Mahler et al. Adv. Mater. 18: 1365-1370, Rachmiel, D., Anconina, I., Rudnick-Glick, S., Halperin-Sternfeld, M., Adler-Abramovich, L. & Sitt, A. (2021) Hyaluronic Acid and a Short Peptide Improve the Performance of a PCL Electrospun Fibrous Scaffold Designed for Bone Tissue Engineering Applications. Int. J. Mol. Sci. 22.

### SUMMARY OF THE INVENTION

According to an aspect of some embodiments of the present invention there is provided a method of inducing bone regeneration in a subject in need thereof, the method comprising administering to the subject a composition comprising a hybrid hydrogel comprising a biocompatible polymer and a plurality of self-assembled peptides, thereby inducing bone regeneration, wherein: each peptide in the plurality of peptides is a dipeptide which comprises at least one aromatic amino acid residue; and the biocompatible polymer is or comprises hyaluronic acid having an average molecular weight of at least 100, at least 500, or at least 1,000 kDa.

According to an aspect of some embodiments of the present invention there is provided a composition for use in inducing bone regeneration in a subject in need thereof, the composition comprising a hybrid hydrogel comprising a biocompatible polymer and a plurality of self-assembled peptides, wherein: each peptide in the plurality of peptides is a dipeptide which comprises at least one aromatic amino acid residue; and the biocompatible polymer is or comprises hyaluronic acid having an average molecular weight of at least 100, at least 500, or at least 1,000 kDa.

According to an aspect of some embodiments of the present invention there is provided a method of inducing bone regeneration, the method comprising contacting bone-forming stem cells and/or osteoblasts differentiated and/or proliferated therefrom with a composition comprising a hybrid hydrogel comprising a biocompatible polymer and a plurality of self-assembled peptides, thereby inducing bone regeneration, wherein: each peptide in the plurality of peptides is a dipeptide which comprises at least one aromatic amino acid residue; and the biocompatible polymer is or comprises hyaluronic acid having an average molecular weight of at least 100, at least 500, or at least 1,000 kDa.

According to some of any of the embodiments described herein, the bone progenitor cells comprise mesenchymal stem cell, macrophages, preosteoblast or lymphocytes.

According to some of any of the embodiments described herein, the biopolymer binds CD44 and TLR2/4 on macrophages.

According to some of any of the embodiments described herein, the contacting is performed ex-vivo.

According to some of any of the embodiments described herein, the contacting is performed in-vivo.

According to some of any of the embodiments described herein, the administering does not comprise administering cells and/or growth factors.

According to some of any of the embodiments described herein, the composition does not comprise cells and/or growth factors.

According to some of any of the embodiments described herein, the composition further comprises an agent selected from the group consisting of an osteo-inductive agent, an anti-inflammatory agent and antibiotics.

According to some of any of the embodiments described herein, the osteo-inductive drug comprises a proteinaceous agent.

According to some of any of the embodiments described herein, the proteinaceous agent is selected from the group consisting of include morphogenic protein (BMP), transforming growth factor-β protein (TGF-β), insulin type growth factor (IGF), and fibroblast growth factor (FGF), specifically, basic fibroblast growth factor (bFGF) or Vascular Endotherial Growth Factor (VEGF).

According to some of any of the embodiments described herein, the subject suffers from a medical condition associated with a bone fracture and/or bone defect.

According to some of any of the embodiments described herein, the medical condition is a fracture.

According to some of any of the embodiments described herein, the medical condition is a fracture non-union.

According to some of any of the embodiments described herein, the bone fracture is a stress fracture.

According to some of any of the embodiments described herein, the medical condition is resorption of alveolar bone.

According to some of any of the embodiments described herein, the resorption of alveolar bone is associated with a missing tooth.

According to some of any of the embodiments described herein, the medical condition is a bone disease.

According to some of any of the embodiments described herein, the bone disease is selected from the group consisting of osteoporosis, osteoporotic fractures, diabetic fractures, bone defects, osteoplastic insufficiency, osteomalacia and resulting fractures, fracture defects, bone growth around graft correction, bone growth disorders, bone tumors, nonunion fractures and hereditary.

According to some of any of the embodiments described herein, the hybrid hydrogel forms upon contacting the plurality of peptides and the biocompatible polymer in an aqueous solution. Preferably the contacting is of an aqueous solution of the plurality of the peptides with an aqueous solution that comprises the biopolymer. Preferably, the aqueous solution that comprises the peptides further comprises a water-immiscible organic solvent in an amount or concentration as described herein.

According to some of any of the embodiments described herein, a total concentration of the peptides and the biocompatible polymer in the aqueous solution is 5 mg/mg.

According to some of any of the embodiments described herein, each of the dipeptides consists of aromatic amino acid residues.

According to some of any of the embodiments described herein, each dipeptide in the plurality of dipeptides is a homodipeptide.

According to some of any of the embodiments described herein, each of the dipeptides is phenylalanine-phenylalanine dipeptide (Phe-Phe).

According to some of any of the embodiments described herein, each dipeptide in the plurality of peptides is an end-capping modified peptide.

According to some of any of the embodiments described herein, each of the end-capping modified dipeptides comprises an aromatic end-capping moiety.

According to some of any of the embodiments described herein, the aromatic end capping moiety is 9-fluorenylmethyloxycarbonyl (Fmoc).

According to some of any of the embodiments described herein, a weight ratio of the dipeptides and the polymer in the aqueous solution ranges from 10:1 to 1:10, or from 5:1 to 1:5, or from 5:1 to 1:1, or from 5:1 to 2:1, or is about 3:1.

According to some of any of the embodiments described herein, the hybrid hydrogel or the composition comprising same is characterized by a storage modulus G′ higher than 1,000 kPa, or higher than 2,000 kPa, or higher than 3,000 kPa, or higher than 4,000 kPa, at 5 Hz frequency and at 25° C.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G show FmocFF/HA composite hydrogel formation and structural characterization. (FIG. 1A) Molecular structure of the FmocFF peptide. (FIG. 1B) Molecular structure of hyaluronic acid. (FIG. 1C) Inverted vials of FmocFF (left), FmocFF/HA composite hydrogel (middle), and HA (right). (FIG. 1D) TEM micrograph of the FmocFF/HA composite hydrogel. (FIG. 1E) SEM micrograph of the FmocFF/HA composite hydrogel. (FIG. 1F) The FmocFF/HA composite hydrogel was formed in a 9 mm diameter×2 mm depth silicone mold. (FIG. 1G) The FmocFF/HA composite hydrogel is injected through a 27-gauge needle.

FIGS. 2A-G show rheological and FTIR analyses of FmocFF/HA composite hydrogel. (FIG. 2A) FTIR spectra of FmocFF/HA, FmocFF and HA. (FIG. 2B) In situ time sweep oscillation measurements of the FmocFF, HA, and the composite FmocFF/HA hydrogel. (FIG. 2C) Viscosity versus shear rate of the FmocFF/HA composite hydrogel. (FIG. 2D) Time sweep when alternate step strain switched from 0.1% to 100%. (FIGS. 2E-F) Digital images demonstrating the self-healing properties of separate layers of the composite hydrogel. (FIG. 2G) Inverted test-tube showing the self-healing property of the hydrogel after mechanical breaking.

FIGS. 3A-H show migration and osteogenic differentiation of MC3T3-E1 preosteoblasts on the hydrogel. (FIG. 3A) Migration of MC3T3-E1 preosteoblasts one day post seeding using confocal microscopy. (FIG. 3B) Optic microscope images of MC3T3-E1 preosteoblasts stained with Alizarin red 14 days after seeding on FmocFF/HA hydrogel with osteogenic media. (FIG. 3C) Optic microscope images of MC3T3-E1 preosteoblasts stained with Alizarin red 14 days after seeding on FmocFF/HA hydrogel with culture media. (FIG. 3D) Optic microscope images of MC3T3-E1 preosteoblasts stained with Alizarin red 14 days after seeding on the plate with osteogenic media. (FIG. 3E) Optic microscope images of MC3T3-E1 preosteoblasts stained with Alizarin red 14 days after seeding on the plate with culture media. (FIG. 3F) Normalized Alizarin red staining absorbance values 14 days after MC3T3-E1 osteogenic differentiation with and without osteogenic media. (FIG. 3G) Normalized alkaline phosphatase (ALP) activity of MC3T3-E1 preosteoblasts 14 days after seeding with and without osteogenic differentiation. (FIG. 3H) Quantification of calcium content in the supernatant of the FmocFF/HA hydrogel 14 days after seeding with and without osteogenic media. Data analyzed using a two-tailed Student's t-test. *p<0.05, **p<0.01.

FIGS. 4A-D show in vivo assessment of the FmocFF/HA composite hydrogel in critical-sized bone defects using Micro-CT at eight weeks. (FIG. 4A) Representative μCT reconstructions of rat calvarial defects in the four groups (FmocFF/HA, Unfilled, BBM, and FmocFF/Alg). A color map representing the thickness of the repaired defect (right side of the calvaria) compared to the corresponding untreated control (left side of calvaria) was made on top view images of the calvarias. Red color indicates a high thickness value, while blue indicates low thickness values. A coronal section of each defect was made to compare the quality of defect fill in the experimental and untreated control group. (FIG. 4B) Mean restored bone volume in the four groups. The dark green part of the columns represents new bone and the light green represents residual graft particles. (FIG. 4C) Mean restored bone volume divided into three areas with equal volumes, namely inner, middle, and outer. The light green part of the columns in the BBM group represents residual graft particles. (FIG. 4D) Mean restored bone mineral density in the four groups relative to the density in the untreated control. Data analyzed using two-way ANOVA with Bonferroni post-hoc test and one-way ANOVA with Tukey's post-test, *p<0.05.

FIGS. 5A-P show histologic sections of the bone defect repair eight weeks post-surgery. (FIG. 5A) H&E staining of the unfilled defect. Bone emerging from the defect margins is seen on both sides (orange arrows). (FIG. 5B) Masson's trichrome staining of the unfilled defect. Bone emerging from the defect margin is seen on the left (orange arrow). (FIG. 5C) High magnification of a Masson's trichrome staining of the unfilled defect. (FIG. 5D). Demonstration of blood vessels in the newly formed bone in an unfilled defect (black arrows). (FIG. 5E) H&E staining of the FmocFF/HA hydrogel-filled defect shows bone emerging from the defect margins as well as bone islands (orange arrows). (FIG. 5F) Masson's trichrome staining of the FmocFF/HA hydrogel-filled defect shows bone islands (orange arrows). (FIG. 5G) High magnification of a Masson's trichrome staining of the FmocFF/HA hydrogel-filled defect showing a bone island containing osteocytes (orange arrow). (FIG. 5H) Demonstration of blood vessels in the newly formed bone in a defect filled with FmocFF/HA hydrogel (black arrows). (FIG. 5I) H&E staining of the defect filled with BBM. Bone emerging from the defect margin is seen on the left (orange arrows). (FIG. 5J) Masson's trichrome staining of the defect filled with BBM showing new bone surrounding the BBM particles (orange arrows). (FIG. 5K) High magnification of a Masson's trichrome staining of the defect filled with BBM (orange arrows showing new bone formation). (FIG. 5L) Blood vessels formed in the new bone surrounding the BBM particles (black arrows). (FIG. 5M) H&E staining of FmocFF/Alg-filled defect. Bone emerging from the defect margins is seen on both sides (orange arrows). (FIG. 5N) Masson's trichrome staining of FmocFF/Alg-filled defect. Bone emerging from the defect margin is seen on the right (orange arrow). (FIG. 5O) High magnification of a Masson's trichrome staining of FmocFF/Alg-filled defect. (FIG. 5P) Demonstration of blood vessels in the newly formed bone in FmocFF/Alg-filled defect (black arrows).

FIGS. 6A-V show immunomodulation of the FmocFF/HA hydrogel one and three weeks after implantation (FIG. 6A) H&E staining of the FmocFF/HA-filled defect one week after implantation showing initial bone formation (orange arrows). (FIG. 6B) Immunohistochemical staining for CD163 one week after FmocFF/HA hydrogel implantation. Brown staining indicating M2 macrophages lining at the hydrogel-periosteum interface (blue arrows). (FIG. 6C) Immunohistochemical staining for CD68 (M1 macrophages, brown staining) one week after FmocFF/HA hydrogel implantation. (FIG. 6D) H&E staining of the FmocFF/HA-filled defect three weeks after implantation showing new bone formation (orange arrows). (FIG. 6E) Immunohistochemical staining for CD163 (M2 macrophages) three weeks after FmocFF/HA hydrogel implantation showing brown staining of cells along the regenerating tissue (blue arrows). (FIG. 6F) Immunohistochemical staining for CD68 (M1 macrophages) three weeks after FmocFF/HA hydrogel implantation. (FIG. 6G) H&E staining of the unfilled defect one week after surgery (Orange arrows showing new bone). (FIG. 6H) Immunohistochemical staining for CD163 (M2 macrophages, brown staining) in the unfilled defect one week after surgery (Blue arrows showing M2 macrophages). (FIG. 6I) Immunohistochemical staining for CD68 (M1 macrophages, brown staining) in the unfilled defect one week after surgery. (FIG. 6J) H&E staining of the unfilled defect three weeks after surgery showing initial bone formation (orange arrows). Immunohistochemical staining for CD163 (M2 macrophages) in the unfilled defect three weeks after surgery (blue arrows). (FIG. 6L) Immunohistochemical staining for CD68 (M1 macrophages) in the unfilled defect three weeks after surgery. (FIG. 6M) H&E staining of the BBM-filled defect one week after surgery. (FIG. 6N) Immunohistochemical staining for CD163 (blue arrows) one week after surgery. (FIG. 6O) Immunohistochemical staining for CD68 (M1 macrophages, brown staining) of the BBM-filled defect one week after implantation. (FIG. 6P) H&E staining of the BBM-filled defect three weeks after implantation (Orange arrows showing new bone). (FIG. 6Q) Immunohistochemical staining for CD163 (M2 macrophages) of the BBM-filled defect three weeks after implantation (blue arrows). (FIG. 6R) Immunohistochemical staining for CD68 (M1 macrophages) of the BBM-filled defect three weeks after implantation. (FIG. 6S) Quantification of CD68 positive macrophages at 1 week and 3 weeks and comparison between the three groups (FmocFF/HA, BBM, and unfilled defects.) (FIG. 6T) Quantification of CD163 positive cells at 1 and 3 weeks and comparison between the three groups (FmocFF/HA, BBM, and unfilled defects.) (FIG. 6U-V) Schematic illustration of the immunomodulation induced by the hydrogel one (FIG. 6U) and three (FIG. 6V) weeks after implantation (purple cells=resident macrophages, M0 macrophages; green cells=M1 macrophages; red cells=M2 macrophages).

### DESCRIPTION OF SPECIFIC EMBODIMENTS OF THE INVENTION

The present invention, in some embodiments thereof, relates to therapy and. More particularly, but not exclusively, to compositions comprising hybrid hydrogels made of self-assembled peptide(s) and biocompatible polymer(s) for use in bone regeneration.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Biomimetic materials that can stimulate and accelerate bone formation without pre-embedding cells and growth factors are of great importance in bone tissue engineering.

In a search for such biomimetic material, the present invention have studied the use of an exemplary hybrid hydrogel formed of a self-assembled peptide structure and a biocompatible polymer, a hybrid FmocFF/HA hydrogel, and have designed compositions containing such a hybrid hydrogel which performs as a stiff ECM-biomimetic hydrogel, and supports complete bone regeneration to the original thickness and density. The hybrid hydrogel is endowed with osteoimmunomodulatory features leading to macrophage recruitment to the hydrogel where they differentiate early into M2 macrophages that promote angiogenesis and osteogenesis.

As is shown in the Examples section which follows, both in vitro and in vivo, an osteo-regenerative effect of the exemplary FmocFF/HA hydrogel is demonstrated. Incorporating the self-assembling peptide, e.g., FmocFF, into the polymeric (e.g., High MW HA) matrix resulted in forming a stiff fibrous hydrogel with a storage modulus value that is beneficial for promoting bone regeneration without the need for inorganic bone ceramics for reinforcement (see, Example 2). The resemblance of the hydrogel to the native bone ECM supported MC3T3-E1 preosteoblast osteodifferentiation (see, Example 4). In vivo, eight weeks after implantation into a rat calvarial critical-sized bone defect, the hydrogel induced bone formation of approximately 95% of the original volume, 2-fold higher than seen either in unfilled defects or in defects filled with a xenogenic bone graft (Example 5). The hydrogel not only induced bone deposition from the defect margins but also created bony islets in the central part of the defect. These two patterns of bone formation resulted in the complete restoration of the original thickness of the calvaria and its original bone density. Without being bound by theory, it is suggested that the hydrogel served as a 3D matrix that maintained the space over time and was then gradually degraded while promoting the host's natural bone healing process by modulating the local immune environment in favor of angiogenesis, osteogenesis, and the osteointegration of the implanted hydrogel.

The simple production, relatively low cost, and ease of handling and delivery, both as an injectable hydrogel and as a custom-made construct, demonstrate the advantageous use of hybrid hydrogels as described herein in various clinical applications in bone regenerative medicine.

Thus, according to an aspect of some embodiments of the invention there is provided a method of inducing bone regeneration, the method comprising contacting bone-forming stem cells and/or osteoblasts differentiated and/or proliferated therefrom with a composition comprising a hybrid hydrogel as described herein, thereby inducing bone regeneration.

According to an aspect of some embodiments of the invention there is provided a composition comprising a hybrid hydrogel as described herein in any of the respective embodiments and any combination thereof, for use in inducing bone regeneration. According to some embodiments, inducing bone regeneration is effected by contacting bone-forming stem cells and/or osteoblasts differentiated and/or proliferated therefrom with the composition comprising the hybrid hydrogel, thereby inducing bone regeneration.

According to an aspect of some embodiments of the invention there is provided a use of a composition comprising a hybrid hydrogel as described herein in any of the respective embodiments and any combination thereof, in the manufacture of a medicament for use in inducing bone regeneration. According to some embodiments, inducing bone regeneration is effected by contacting bone-forming stem cells and/or osteoblasts differentiated and/or proliferated therefrom with the composition comprising the hybrid hydrogel, thereby inducing bone regeneration.

As used herein “bone” refers to any bone of a vertebrate. These include long bones (e.g. femur, humerus, phalanges), short bones (e.g. carpals, tarsals), flat bones (e.g. cranium, ribs, scapula, sternum, pelvic girdle), irregular bones (e.g. vertebrae), sesamoid bones (e.g. patella).

According to a specific embodiment, the bone is a long bone.

As used herein “bone regeneration” refers to the process of production of a bone tissue using the hydrogel described herein as a scaffold or carrier. A bone tissue is typically composed of:
Periosteum—the dense, tough outer shell that contains blood vessels (it may comprise nerves if such cells are present at least in the ex vivo settings);
Compact or dense tissue—the hard, smooth layer that protects the tissue within;
Spongy or cancellous tissue—the porous, honeycombed material found inside most bones, which allows the bone to be strong yet lightweight.
The bone marrow—the jelly-like substance found inside the cavities of some bones (including the pelvis) that produces blood cells is considered a different tissue than the bone itself.

Key cells that make up the bone include osteogenic, osteoblasts, osteocytes, and osteoclasts (together recognized as the basic multicellular unit (BMU)).

According to a specific embodiment, the contacting is performed ex-vivo.

In such a case measures are taken to include the relevant cells which can form at least a bone but preferably also a vascularized bone.

Examples of such cells include, but are not limited to, chondrocytes, bone marrow cells, osteocytes, periosteal cells, perichondrial cells, fibroblasts, mesenchymal cell, mesenchymal stem cell, adipose derived cells, adipose derived stem cells, neuronal cells, hippocampal cells, epidermal cells, endothelial cells, epithelial cells, spinous cells, granular cells or precursors (progenitors) of any of the foregoing), embryonic stem cells or precursors (progenitors) of any of the foregoing), or immune cells which can augment the process e.g., of vascularization and support an immune niche e.g., neutrophils, lymphocytes, macrophages, dendritic cells, or precursors (progenitors) of any of the foregoing).

According to a specific embodiment, the cells are stem cells or bone forming progenitor cells.

According to a specific embodiment, the cells are mesenchymal stem cells.

According to a specific embodiment, the cells are preosteoblasts.

According to a specific embodiment, the cells are differentiated cells.

According to a specific embodiment, the cells are osteoblasts.

According to a specific embodiment, the cells are of more than one type, e.g., 2, 3, 4 or more.

Methods of assessing cell proliferation and differentiation are well known in the art. Bone qualification methods are shown in the Examples section which follows.

According to a specific embodiment, the contacting is performed in-vivo.

The regenerative function of the hydrogel as shown in Example 5 can be harnessed towards therapeutic applications.

Thus, according to an aspect of the invention there is provided a method of inducing bone regeneration in a subject in need thereof, the method comprising administering to the subject a composition comprising a hybrid hydrogel as described herein in any of the respective embodiments and any combination thereof, thereby inducing bone regeneration.

According to an alternative or an additional aspect, there is provided a composition comprising a hybrid hydrogel as described herein in any of the respective embodiments and any combination thereof for use in inducing bone regeneration in a subject in need thereof.

According to an alternative or an additional aspect, there is provided a use of a composition comprising a hybrid hydrogel as described herein in any of the respective embodiments and any combination thereof in the manufacture of a medicament for use in inducing bone regeneration in a subject in need thereof.

As used herein, the terms “treating” and “treatment” include abrogating, substantially inhibiting, slowing or reversing the progression of a condition, substantially ameliorating clinical or aesthetical symptoms of a condition or substantially preventing the appearance of clinical or aesthetical symptoms of a condition.

Herein, the phrase “medical condition associated with a bone fracture and/or a bone defect” encompasses bone fractures and/or bone defects per se, as well as medical conditions which may lead to formation of a bone fracture and/or defect (e.g., resorption of bone) and medical conditions which may result from a bone fracture and/or defect (e.g., a complication of a bone fracture and/or defect).

Herein, the phrase “bone fracture” encompasses any break in the continuity of a bone, including, for example, incomplete fractures (wherein the bone fragments are still partially joined), complete fractures (wherein bone fragments separate completely) and comminuted fractures (wherein a bone breaks into several pieces). The mechanism of bone fracture may be, for example, traumatic fracture (e.g., caused by high force impact or stress), pathological fracture (i.e., associated with a medical condition which weakens the bones, such as osteoporosis, bone cancer or osteogenesis imperfecta) or periprosthetic fracture (i.e., associated with mechanical weakness adjacent to an implant).

In the context of medical conditions associated with bone fractures, the terms “treating” and “treatment” encompass, for example, substantially healing, at least in part, a bone fracture (e.g., a fracture non-union which does not heal without intervention), substantially increasing a rate at which a bone fracture heals, substantially ameliorating or preventing the appearance of symptoms of a bone fracture (e.g., pain, loss of functionality of a portion of the body, defective bone formation), and preventing or reducing the likelihood of a bone fracture occurring, for example, due to a medical condition (e.g., prophylaxis). Treatment of a bone fracture as described herein may optionally be performed in combination with standard treatments of bone fractures, such as immobilization of bones (e.g., with a cast) and/or surgery.

In some embodiments according to any of the aspects of embodiments described herein, the method or treatment is for treating a fracture non-union (e.g., the medical condition is a fracture non-union).

Herein and in the art, the phrase “fracture non-union” refers to a medical condition in which a bone fracture is present, and there is no reasonable expectation that the fracture will heal without intervention.

The skilled person will be readily capable of determining a presence of a fracture non-union.

In some embodiments according to any of the aspects of embodiments relating to non-unions, a fracture non-union is determined based on non-consolidation at the fracture site 6 months after the fracture was formed, and/or based on an absence of progress in callus formation at the fracture site at 4 week intervals (e.g., as described by Giannotti et al. [Clin Cases Miner Bone Metab 2013, 10:116-120]).

In some embodiments according to any of the aspects of embodiments described herein, the bone fracture is a stress fracture, that is, a bone fracture caused by repeated stress over time (e.g., by running and/or jumping), and the medical condition is any medical condition associated with a stress fracture (optionally the medical condition is a stress fracture per se).

In some embodiments, treating a medical condition associated with a stress fracture comprises increasing a rate at which an existing stress fracture heals.

In some embodiments, treating a medical condition associated with a stress fracture comprises reducing the likelihood of a stress fracture occurring, for example, in a subject susceptible to stress fractures. Examples of subjects susceptible to stress fractures include, without limitation, athletes, runners, soldiers and other people subject to considerable physical exercise.

Herein, the phrase “bone defect” encompasses any missing portion of a bone, including, bone missing due to trauma (e.g., wherein a bone fracture results in a missing bone fragment), surgery (e.g., wherein bone is surgically removed in order to remove cancer cells), resorption of bone, an acquired medical condition (e.g., wherein an acquired medical condition causes a portion of a bone to disappear via resorption) and/or congenital conditions (e.g., wherein a congenitally misshapen bone is associated with one or more defects in the bone structure), a space between a bone and an implant intended to be osseointegrated with the bone (including, but not limited to, an implant anchored in a bone, for example, via a bolt or screw).

Examples of medical conditions involving resorption of bone include, without limitation, bone resorption associated with inflammatory conditions (e.g., periodontitis), which may comprise resorption of bone near the site of inflammation, and resorption of alveolar bone associated with a missing tooth.

Herein, the terms “osseointegration” and “osseointegrated” refer to formation of a direct structural connection (e.g., without intervening connective tissue) between living bone and an implant; and includes, but is not limited to, growth of bone into an implant (e.g., a porous implant), a process also known in the art as “osseoincorporation”.

In the context of medical conditions associated with bone defects, the terms “treating” and “treatment” encompass, for example, substantially healing, at least in part, a bone defect (e.g., replacement of at least a portion of missing bone by bone regeneration), substantially increasing a rate at which a bone defect heals (e.g., a rate of bone regeneration), substantially ameliorating or preventing the appearance of symptoms of a bone defect (e.g., pain, loss of functionality of a portion of the body, defective bone formation), and preventing or reducing formation of a bone defect (e.g., prophylaxis), for example, formation of a bone defect by bone resorption. Treatment of a bone defect as described herein may optionally be performed in combination with standard treatments of the respective bone defect.

In some embodiments according to any of the aspects of embodiments described herein, the medical condition is resorption of alveolar bone. In some of these embodiments, the method or treatment is for preserving and/or regenerating alveolar bone. Examples of resorption of alveolar bone include, without limitation, resorption associated with a missing tooth and resorption associated with inflammation (e.g., periodontitis).

In some embodiments, the method or treatment or use is for preserving and/or regenerating alveolar bone surrounding a dental implant (e.g., a dental implant which comprises or supports a prosthetic tooth, crown, dental bridge and/or fixed denture), for example, to hold the dental implant in place, thereby increasing the utility of the implant and/or the likelihood of success of the dental implantation. In some embodiments, the method or treatment is effected following implantation of a dental implant, for example, in order to promote regeneration of alveolar bone (e.g., alveolar bone characterized by a bone defect associated with resorption of the bone due to a missing tooth and/or periodontitis). In alternative or additional embodiments, the method or treatment is effected prior to implantation of a dental implant, for example, in order to preserve alveolar bone by preventing or reducing alveolar bone resorption (e.g., upon loss of a tooth, when a significant amount of time is expected to pass before implantation of a dental implant.

In some embodiments according to any of the aspects of embodiments described herein, the bone defect is in the skull (cranium or lower jawbone). In some embodiments, the bone defect is a calvarial bone defect.

Without being bound by any particular theory, it is believed that bone in the skull (e.g., in the calvaria) is particularly susceptible to poor healing of bone defects, in which promotion of bone growth would be advantageous.

In some embodiments according to any of the aspects of embodiments described herein, the method and/or treatment and/or use comprises promoting osseointegration of an implant, for example, by promoting bone growth in a space between a bone (e.g., calvarial bone) and the implant. The medical condition may optionally be any medical condition for which osseointegration of an implant is beneficial.

Examples of implants for which osseointegration may be promoted include, without limitation, dental implants, bone grafts (e.g., bone allografts), chin implants, craniofacial prostheses (e.g., artificial ears, eyes and/or noses), bone-anchored limb prostheses, bone-anchored hearing aids, and joint prostheses (e.g., for hip and/or knee replacement).

In some embodiments of any of the embodiments described herein, the bone fracture and/or bone defect is not associated with osteoporosis (e.g., the medical condition is not osteoporosis).

The ability of the hydrogel per se to promote bone regeneration negates the need to include cells and/or growth factors.

Thus, according to some embodiments of the invention, administering does not comprise administering cells and/or growth factors at any stage of the method, i.e., prior to, following or concomitant with administration of the scaffold.

According to some embodiments the composition comprising the hybrid hydrogel does not comprise cells and/or growth factors, e.g., as part of the formulation.

According to other embodiments, the composition further comprises one or more therapeutically active agents. Exemplary therapeutically active agents include, but are not limited to, an osteo-inductive agent (e.g., polymers e.g., proteins, ceramics, metals, e.g., TI), anti-inflammatory agents and antibiotics.

A detailed description of osteo-inductive materials can be found in European Cells and Materials Vol. 21 2011 (pages 407-429) AMC Barradas et al. Osteoinductive biomaterials: current knowledge.

According to a specific embodiment, the osteo-inductive drug comprises a proteinaceous agent.

Exemplary proteinaceous agents include, but are not limited to, morphogenic protein (BMP), transforming growth factor-β protein (TGF-β), insulin type growth factor (IGF), and fibroblast growth factor (FGF), specifically, basic fibroblast growth factor (bFGF) or Vascular Endotherial Growth Factor (VEGF).

A composition as described herein in any of the respective embodiments comprises a hydrogel (a hybrid hydrogel) which comprises a fibrous network of a plurality of self-assembled peptides and a biocompatible polymer.

As used herein, and is well-known in the art, the term “hydrogel” refers to a material that comprises one or more solid, typically fibrous, networks formed, at least in part, of water-soluble natural or synthetic polymer chains, and typically containing at least 50%, or at least 70%, or at least 80%, or at least 90%, or at least 95%, water.

As used herein the phrase “fibrous network” refers to a set of connections formed between the plurality of fibrous components. Herein, the fibrous components are composed, at least in part, of peptide fibrils, each formed upon self-assembly of short peptide building blocks, as is further detailed hereinbelow.

Since the hydrogels described herein are formed of two or more types of components, the hydrogels are also referred to herein interchangeably as “hydrogel hybrid” or “hybrid hydrogel” or “composite” or “hydrogel composite” or “hybrid hydrogel composite” or “hydrogel hybrid composite”. According to some embodiments, the hybrid hydrogel is a physical hybrid, in which interaction between the self-assembled peptides and the polymer includes physical interactions, such that these components are not covalently attached to one another.

As currently accepted in the art, the term “biocompatible” is generally defined as “the ability of a material to perform with an appropriate host response in a specific application” [see, The Williams dictionary of Biomaterials].

In the context of biomaterial applications such as bone regeneration, biocompatibility refers to the ability to perform as a supportive matrix to an appropriate cellular activity, without eliciting any undesirable effects in those cells, or inducing any undesirable local or systemic responses in the host.

In the context of embodiments of the present invention, a “biocompatible material” describes a material (e.g., a natural or synthetic polymer) or matrix (e.g., hydrogel or scaffold) that does not interfere, and preferably provides a suitable environment for, cellular activity.

A “cellular activity” includes, for example, cell viability, cell growth (proliferation), cell differentiation, cell migration, cell adhesion, molecular and mechanical signaling systems, and fluid transport through cells or a tissue so as to allow nutritive environment.

The biocompatibility of a substance can be determined by methods well known in the art, following the definitions hereinabove and international guidelines, using widely recognized safety assays. Optionally, biocompatible substances can be selected from existing lists of such substances.

In some embodiments, the biocompatible polymer is water-soluble.

Exemplary biocompatible polymers that are suitable for use in the context of embodiments of the present invention are polysaccharides.

The term “polysaccharide” as used herein is meant to include compounds composed of 10 saccharide units and up to hundreds and even thousands of monosaccharide units per molecule, which are held together by glycoside bonds and range in their molecular weights from around 5,000 and up to millions of Daltons.

Polysaccharides that have desired physical properties, as defined herein with respect to swelling capability and viscoelasticity, are typically highly hydrated polysaccharides, or highly hydrated linear polysaccharides, as described hereinabove.

In some embodiments, the polysaccharide is a GAG. As noted hereinabove, GAGs are natural polymers which are major components of the native extracellular matrix (ECM), and are known to support enhanced cell attachment and proliferation.

In some embodiments, the polysaccharide is hyaluronic acid (HA).

The biocompatible polymers can be a natural polymer or a synthetic polymer.

In some embodiments, the biocompatible polymer of choice has an average molecular weight of at least 100 kDa or of at least 1000 kDa or of at least 2000 kDa. Polymers featuring such an average molecular weight are also referred to herein and in the art as high molecular weight polymers. In some embodiments, the biocompatible polymer has an average molecular weight that ranges from 100 kDa to 10,000 kDa, or from 1,000 kDa to 10,000 kDa, for example, from 1,000 kDa to 5,000 kDa, or from 2,000 to 5,000 kDa, including any intermediate values and subranges therebetween.

In some embodiments, the hybrid hydrogel comprises more than one type of a biocompatible polymer as described herein.

Without being bound by any particular theory, the hybrid hydrogels described herein comprise fibrous networks which can be composed of peptide fibrils formed upon self-assembly of the peptides, with the biocompatible polymer being encaged therewithin, or, alternatively or in addition, can be composed of fibrous peptide structures having entangled therewith the polymer.

In some embodiments, the biocompatible polymer is hyaluronic acid (HA) having an average molecular weight (being high MW hyaluronic acid) of at least 100 kDa, preferably of at least 500 kDa, or at least 1,000 kDa or at least 2,000 kDa, e.g., of from 100 kDa to 100,000 kDa, or from 1,000 kDa to 100,000 kDa, for example, from 1,000 kDa to 50,000 kDa, or from 1,000 kDa to 20,000 kDa, or from 1,000 kDa to 10,000 kDa, or from 1,000 kDa to 5,000 kDa, or from 2,000 kDa to 5,000 kDa, including any intermediate values and subranges therebetween.

As discussed in the Background section hereinabove, it is suggested that high molecular weight (MW; Mw) hyaluronic acid is capable of functioning as a polarizing agent (see Rayahin et al. 2015 CS Biomater. Sci. Eng. 2015, 1, 7, 481-493 “High and Low Molecular Weight Hyaluronic Acid Differentially Influence Macrophage Activation”), yet, its activity is limited by its fast biodegradation, which occurs before it binds to the macrophages. As shown herein, it has been surprisingly uncovered the hybridization with self-assembled peptides as described herein, prolongs the life time of the high MW HA, while maintaining its polarizing activity.

Thus, according to a specific embodiment, the biocompatible polymer (e.g., biopolymer) binds CD44 and TLR2/4 on macrophages, e.g., M1.

Without being bound by theory, the CD44 gets activated and promotes bone regeneration, whilst the TLR2/4 gets inhibited upon said binding.

According to some embodiments of the present invention, the fibrils composing the hydrogel have an average diameter or a cross-section of less than 1 km. In some embodiments, the fibrils have an average diameter that ranges from about 1 nm to about 500 nm, more preferably from about 10 nm to about 500 nm, more preferably from about 10 nm to about 200 nm and more preferably from about 10 nm to about 100 nm.

As used herein, the phrase “a plurality of self-assembled peptides” encompasses any peptides that under certain conditions (e.g., concentration and/or temperature), spontaneously rearrange in an aqueous solution so as to form peptide fibrils that form the hydrogel's fibrous network in the solution.

In some embodiments, the peptides used for forming the hybrid hydrogels described herein each independently has at least two amino acid residues and up to 6 amino acid residues, provided that at least one amino acid residue, in each peptide of the plurality of peptides used, is an aromatic amino acid. Thus, each of the peptides used for forming the hydrogels described herein can have two, three, four, five or six amino acid residues, with one or more aromatic amino acid residues as described herein.

Each peptide in the plurality of peptides used for forming the hydrogel comprises at least one aromatic amino acid residue

In some embodiments of the present invention, at least one peptide in the plurality of peptides used for forming the hydrogel is a polyaromatic peptide, comprising two or more aromatic amino acid residues. In some embodiments, at least one peptide in the plurality of peptides consists essentially of aromatic amino acid residues. In some embodiments, each peptide in the plurality of peptides consists essentially of aromatic amino acid residues.

Thus, for example, the peptides used for forming the hybrid hydrogel described herein can include any combination of: dipeptides composed of one or two aromatic amino acid residues; tripeptides including one, two or three aromatic amino acid residues; tetrapeptides including two, three or four aromatic amino acid residues; pentapeptides including two, three, four or five aromatic amino acid residues; and hexapeptides including two, three, four, five or six aromatic amino acid residues.

In some embodiments, one or more peptides in the plurality of peptides used for forming the hybrid hydrogel include two amino acid residues, and hence is a dipeptide.

In some embodiments, each of the peptides used for forming the hybrid hydrogel comprises two amino acid residues and therefore the hybrid hydrogel is formed from a plurality of dipeptides.

Each of these dipeptides can include one or two aromatic amino acid residues. Preferably, each of these dipeptides includes two aromatic amino acid residues. The aromatic residues composing the dipeptide can be the same, such that the dipeptide is a homodipeptide, or different. In some embodiments, the hydrogel is formed of a plurality of self-assembled homodipeptides (e.g., aromatic homodipepides, each consisting of two aromatic amino acid residues).

Hence, in some embodiments of the present invention, each peptide in the plurality of peptides used for forming the hybrid hydrogel is a homodipeptide composed of two aromatic amino acid residues that are identical with respect to their side-chains residue.

The phrase “aromatic amino acid residue”, as used herein, refers to an amino acid residue that has an aromatic moiety in its side-chain.

As used herein, the phrase “aromatic moiety” describes a monocyclic or polycyclic moiety having a completely conjugated pi-electron system. The aromatic moiety can be an all-carbon moiety or can include one or more heteroatoms such as, for example, nitrogen, sulfur or oxygen. The aromatic moiety can be substituted or unsubstituted, whereby when substituted, the substituent can be, for example, one or more of alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano and amine.

Exemplary aromatic moieties include, but are not limited to, phenyl, biphenyl, naphthalenyl, phenanthrenyl, anthracenyl, [1,10]phenanthrolinyl, indoles, thiophenes, thiazoles and, [2,2′]bipyridinyl, each being optionally substituted. Thus, representative examples of aromatic moieties that can serve as the side chain within the aromatic amino acid residues described herein include, without limitation, substituted or unsubstituted naphthalenyl, substituted or unsubstituted phenanthrenyl, substituted or unsubstituted anthracenyl, substituted or unsubstituted [1,10]phenanthrolinyl, substituted or unsubstituted [2,2′]bipyridinyl, substituted or unsubstituted biphenyl and substituted or unsubstituted phenyl. The aromatic moiety can alternatively be substituted or unsubstituted heteroaryl such as, for example, indole, thiophene, imidazole, oxazole, thiazole, pyrazole, pyridine, pyrimidine, quinoline, isoquinoline, quinazoline, quinoxaline, and purine.

The hybrid hydrogels of the present embodiments can be composed of linear and/or cyclic peptides (e.g., cyclic di-peptides of phenylalanine). In exemplary embodiments, the hybrid hydrogels are formed of linear peptides as described herein.

According to some embodiments of the present invention, one or more, or each, of the peptides in the plurality of peptides that form the hydrogel described herein is an end-capping modified peptide.

The phrase “end-capping modified peptide”, as used herein, refers to a peptide which has been modified at the N-(amine) terminus and/or at the C-(carboxyl) terminus thereof. The end-capping modification refers to the attachment of a chemical moiety to the terminus, so as to form a cap. Such a chemical moiety is referred to herein as an end-capping moiety and is typically also referred to herein and in the art, interchangeably, as a peptide protecting moiety or group.

The phrase “end-capping moiety”, as used herein, refers to a moiety that when attached to the terminus of the peptide, modifies the end-capping. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2.sup.nd ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

Representative examples of C-terminus end-capping moieties are typically moieties that lead to acylation of the carboxy group at the C-terminus and include, but are not limited to, benzyl and trityl ethers as well as alkyl ethers, tetrahydropyranyl ethers, trialkylsilyl ethers, allyl ethers, monomethoxytrityl and dimethoxytrityl. Alternatively the —COOH group of the C-terminus end-capping may be modified to an amide group.

Other end-capping modifications of peptides include replacement of the amine and/or carboxyl with a different moiety, such as hydroxyl, thiol, halide, alkyl, aryl, alkoxy, aryloxy and the like, as these terms are defined herein.

In a preferred embodiment of the present invention, all of the peptides that comprise the hydrogels are end-capping modified only at the N-termini.

However, other combinations of N-terminus end capping and C-terminus end capping of the various peptides composing the hydrogel are also contemplated. These include, for example, the presence of certain percents of end-capping modified peptides within the plurality of peptides, whereby the peptides are modified at the N-termini and/or the C-termini.

Another chemical property of an end-capping of a peptide is its hydrophobic/hydrophilic nature, which when unmodified, is hydrophilic in peptides. Altering the hydrophobic/hydrophilic property of one or both of the end-capping of the peptide may result, for example, in altering the morphology of the resulting fibrous network.

End-capping moieties can be further classified by their aromaticity. Thus, end-capping moieties can be aromatic or non-aromatic.

Representative examples of non-aromatic end capping moieties suitable for N-terminus modification include, without limitation, formyl, acetyl trifluoroacetyl, tert-butoxycarbonyl, trimethylsilyl, and 2-trimethylsilyl-ethanesulfonyl. Representative examples of non-aromatic end capping moieties suitable for C-terminus modification include, without limitation, amides, allyloxycarbonyl, trialkylsilyl ethers and allyl ethers.

Representative examples of aromatic end capping moieties suitable for N-terminus modification include, without limitation, fluorenylmethyloxycarbonyl (Fmoc). Representative examples of aromatic end capping moieties suitable for C-terminus modification include, without limitation, benzyl, benzyloxycarbonyl (Cbz), trityl and substituted trityl groups.

In some embodiments of the present invention, the end-capping modified peptides N-terminus-modified peptides, modified by an aromatic (e.g. Fmoc) end-capping moiety.

The end-capping modified peptides utilized according to the present embodiments can be collectively represented by the following general Formula I:

R₁-[A₁]-[A₂]- . . . [Aₙ]—R₂ Formula I
wherein:
n is an integer from 2 to 6, preferably 2;
A₁, A₂, . . . , Aₙ are each independently an amino acid residue as this term is defined herein, provided that at least one of A₁, A₂, . . . , Aₙ is an aromatic amino acid residue as this term is defined herein;
R₁ is an N-terminus end-capping moiety or absent; and
R₂ is a C-terminus end-capping moiety or absent.

In some embodiments, R₁ is an N-terminus end-capping moiety, preferably an aromatic end-capping moiety, and R₂ is absent—that is, the peptide has at the C-terminus the carboxylic acid of the An amino acid residue.

As described hereinabove, according to some embodiments of the present invention, the hydrogel comprises one or more end-capping modified homodipeptide.

Representative examples of end-capping modified homodipeptides include, without limitation, an end-capping modified naphthylalanine-naphthylalanine dipeptide, phenanthrenylalanine-phenanthrenylalanine dipeptide, anthracenylalanine-anthracenylalanine dipeptide, [1,10]phenanthrolinylalanine-[1,10]phenanthrolinylalanine dipeptide, [2,2′]bipyridinylalanine-[2,2′]bipyridinylalanine dipeptide, (pentahalo-phenylalanine)-(pentahalo-phenylalanine) dipeptide, phenylalanine-phenylalanine dipeptide, (amino-phenylalanine)-(amino-phenylalanine) dipeptide, (dialkylamino-phenylalanine)-(dialkylamino-phenylalanine) dipeptide, (halophenylalanine)-(halophenylalanine) dipeptide, (alkoxy-phenylalanine)-(alkoxy-phenylalanine) dipeptide, (trihalomethyl-phenylalanine)-(trihalomethyl-phenylalanine) dipeptide, (4-phenyl-phenylalanine)-(4-phenyl-phenylalanine) dipeptide and (nitro-phenylalanine)-(nitro-phenylalanine) dipeptide, whereby these homodipeptides are preferably end-capping modified by an aromatic moiety, and more preferably, are end-capping modified at the N-terminus thereof by an aromatic moiety such as Fmoc.

In some embodiments of the present invention, the plurality of peptides in the hybrid hydrogel described herein comprises, or consists essentially of, a plurality of dipeptides, each comprising at least one aromatic amino acid residue.

In some embodiments, in each of the dipeptides both amino acid residues are aromatic amino acid residues, as described herein.

In some embodiments, in each of the dipeptides, the aromatic amino acid residues are the same, such that each of the dipeptides is a homodipeptide, or an aromatic homodipeptide.

In some embodiments, the plurality of dipeptides comprises, or consists essentially of, a plurality of Phe-Phe homodipeptides.

In some embodiments, the plurality of dipeptides comprises, or consists essentially of, a plurality of homodipeptides (e.g., Phe-Phe) which are end-capping modified peptides, wherein the end-capping moiety in such peptides in an aromatic moiety such as Fmoc.

In some embodiments, the plurality of peptides comprises, or consists essentially of, a plurality of Fmoc-Phe-Phe (Fmoc-FF).

In some embodiments, the hybrid hydrogel comprises a fibrous network of a plurality of dipeptides, as described herein (e.g., Fmoc-FF), and hyaluronic acid.

In some embodiments, the hybrid hydrogel comprises a fibrous network of a plurality of dipeptides, as described herein (e.g., Fmoc-FF), and any other biocompatible polymer, as described herein.

In some embodiments, the hybrid hydrogel comprises a fibrous network of a plurality of dipeptides, as described herein (e.g., Fmoc-FF), and hyaluronic acid having a molecular weight of at least 10 kDa, or at least 100 kDa, preferably of at least 1000 kDa, as described herein in the respective embodiments.

A composition as described herein in any of the respective embodiments comprises a hybrid hydrogel as described herein, formed upon contacting the plurality of peptides as described herein the biocompatible polymer as described herein with an aqueous solution, upon dissolving the plurality of peptides in a water-miscible organic solvent. According to some embodiments, the composition comprises an aqueous solution, for example, an aqueous solution in which the hybrid hydrogel forms.

In some embodiments, the hybrid hydrogel is prepared by dissolving the polymer in the aqueous solution and contacting a solution comprising the plurality of peptides with the aqueous solution in which the polymer is dissolved.

In some embodiments, the plurality of peptides is dissolved in a water-miscible solvent, prior to contacting the plurality of peptides with the polymer and the aqueous solution.

Contacting a solution of the peptides dissolved in an organic solvent with an aqueous solution comprising dissolved polymer can be regarded as diluting the peptide's solution to a concentration that allows self-assembly of the peptides.

The phrase “water-miscible organic solvent”, as used herein, refers to organic solvents that are soluble in water. Several factors inherent in the structure of the solvent molecules can affect the miscibility of organic solvents in water, such as for example, the length of the carbon chain and the type of functional groups therein. Hydrogen bonding plays a key role in making organic solvents miscible in water. For example, in alcohols, the hydroxyl group can form hydrogen bonding with water molecules. In addition, aldehydes, ketones and carboxylic acids can form hydrogen bonding via the carbonyl oxygen. Hydrogen bonding between ether and water molecules is also possible, enabling some degree of miscibility of simple ethers in water.

Examples of water-miscible organic solvents include, without limitation, simple alcohols, such as, methanol, ethanol, 1-propanol, 2-propanol, 1-butanol, 2-butanol, 2-methyl-1-propanol, 2-methyl-2-propanol, 2,2-dimethyl-1-propanol and their halogen substituted analogues, ethylene glycol, acetone, dimethylsulfoxide, acetic acid diethyl ether, tetrahydrofuran etc.

Representative examples of organic solvents that were successfully practiced in generating exemplary hydrogels according to the present invention include, acetone, dimethylsulfoxide and hexafluoroisopropanol (e.g., 1,1,1,3,3,3-hexafluoro-2-propanol, abbreviated herein as HFIP).

In some embodiments, the organic solvent is dimethylsulfoxide (abbreviated DMSO).

According to exemplary embodiments, for the purpose of compositions for any of the uses described herein, the total amount of the organic solvent (e.g., DMSO) in the hybrid hydrogel does not exceed 5%, and is preferably lower than 3%, by weight, of the total weight of the hydrogel, or is 2.5% by weight or lower.

The present inventors have surprisingly uncovered that reducing the amount of the water-miscible organic solvent in peptides solution used for forming the hydrogel and hence in the formed hybrid hydrogel allows forming the hybrid hydrogel and results not only in improved biocompatibility due to a reduced amount of the organic solvent but also in hybrid hydrogels that exhibit improved mechanical properties (e.g., G′ values).

In some embodiments, the total concentration of the plurality of peptides and the polymer and in the aqueous solution ranges from about 1 mg/ml to about 50 mg/ml, or from 1 mg/ml to 30 mg/ml, or from 1 mg/ml to 20 mg/ml, or from 1 mg/ml to 10 mg/ml, including any intermediate values and subranges therebetween.

In some embodiments, the total concentration of the plurality of peptides and the polymer and in the aqueous solution is about 5 mg/ml.

The final concentration of the plurality of peptide and of the biocompatible polymer in the aqueous solution, and also of the organic solvent as described herein, and hence in the generated hybrid hydrogel can be readily determined by determining the concentration of each component in the aqueous solution.

For example, one can prepare a stock solution of the peptides in a water-miscible organic solvent or in a solvent mixture of such an organic solvent and water, in a certain concentration of the peptides, and a stock aqueous solution in which the polymer is dissolved in the same concentration of the polymer as for the peptide solution, and then mix the desired relative amounts of the solutions, so as to determine the ratio between the components in hybrid hydrogel. The concentration of each component in the solution will determine the final total concentration of the components in the hybrid hydrogel. Other manipulations of the concentration of each component in its stock solution and of the ratio between the stock solutions when contacted are also contemplated.

In some embodiments, the aqueous solution is a buffer (e.g., PBS).

The process of generating the hybrid hydrogels described hereinabove is preferably performed at room temperature. Alternatively, it can be effected at a physiological temperature (e.g., at 37° C.).

In some embodiments, contacting is further effected by mixing the formed aqueous solution (containing both components). Mixing can be performed, for example, by manual or mechanical shaking (e.g., by vortex), or by magnetic or mechanical stirring. In some embodiments, missing is performed by means of vortex.

Notably, the hybrid hydrogels described herein are generated without using a chemical crosslinking agent or any other chemical or physical reinforcing agents.

Once both the peptides and the polymer are contacted with an aqueous solution, and optionally the solution is mixed, a hybrid hydrogel is formed.

According to some embodiments of the present invention, the preparation of the hydrogel is effected prior to its application to a desired application site. Thus, for example, when the desired application site is a bodily organ or cavity, the hydrogel is prepared ex-vivo, prior to its application, by contacting the plurality of peptides, the polymer and an aqueous solution, as described hereinabove, and is administered subsequent to its formation.

In some embodiments, the total concentration of the peptide content (the plurality of peptides) and the biocompatible polymer in the composition ranges from 1 to 10, or from 1 to 5 weight percent of the total weight of the hybrid hydrogel, including any intermediate values and subranges therebetween, with the balance being water or an aqueous solution (e.g., a buffer).

In some embodiments, the total concentration of the components ranges from 0.5 weight percent to 2.5 weight percent of the total weight of the gel.

In embodiments where the biocompatible polymer is hyaluronic acid, the total concentration of the peptide content (the plurality of peptides) and the biocompatible polymer ranges from 0.5 to 1 weight percent of the total weight of the gel.

According to alternative embodiments, once the hydrogel forms, the composition is dried (e.g., lyophilized), such that composition comprises the fibrous network forms of the plurality of peptides and the biocompatible polymer, and optionally a pharmaceutically acceptable carrier, which is mixed with the dried hydrogel.

According to the present embodiments, a total concentration of the plurality of peptides and the biocompatible hydrogel is 5 mg/ml, in the aqueous formulation in which it is formed. Without being bound by any particular theory, it is assumed that the hybrid hydrogel formed at this concentration provides the mechanical properties required for bone regeneration applications as described herein.

According to some of these embodiments, a weight ratio of the plurality of peptides and the biocompatible polymer can range from 10:1 to 1:10, from 5:1 to 1:5, from 4:1 to 1:4, from 3:1 to 1:3, or from 2:1 to 1:2, or from 5:1 to 1:1, or from 5:1 to 2:1 and in some embodiments it is 3:1.

According to some of any of the embodiments described herein, the hybrid hydrogel comprises a plurality of self-assembled Fmoc-FF peptides and hyaluronic acid (or a salt thereof).

According to some of any of the embodiments described herein, the hybrid hydrogel comprises a plurality of self-assembled Fmoc-FF peptides and hyaluronic acid (or a salt thereof) having an average molecular weight of at least 100 kDa or at least 1000 kDa as described herein.

According to some of any of the embodiments described herein, the hybrid hydrogel comprises a plurality of self-assembled Fmoc-FF peptides and hyaluronic acid (or a salt thereof), wherein a weight ratio therebetween is as described hereinabove (e.g., about 3:1).

According to some of any of the embodiments described herein, the hybrid hydrogel comprises a plurality of self-assembled Fmoc-FF peptides and hyaluronic acid (or a salt thereof), wherein the hybrid hydrogel is formed in an aqueous solution in which a total concentration of the plurality of peptides and the HA or the salt thereof is 5 mg/ml, or as described hereinabove.

According to some of these embodiments, the average MW of the HA of the salt thereof at least 100 kDa or at least 1000 kDa as described herein. According to some of these embodiments, the weight ratio of the plurality of peptides and the HA or the salt thereof is as described hereinabove (e.g., about 3:1).

According to some of any of the embodiments described herein, the hybrid hydrogel comprises a plurality of self-assembled Fmoc-FF peptides and hyaluronic acid (or a salt thereof), wherein the hybrid hydrogel is formed in an aqueous solution in which a total concentration of the plurality of peptides and the HA or the salt thereof is 5 mg/ml, or as described hereinabove, and wherein a weight ratio therebetween is as described hereinabove (e.g., about 3:1). According to some of these embodiments, the average MW of the HA of the salt thereof at least 100 kDa or at least 1000 kDa as described herein.

According to some of any of the embodiments described herein, one or more of a total concentration of plurality of peptides and the biocompatible polymer in the aqueous solution in which it forms, a weight ratio between the plurality of peptides and the biocompatible polymer, an amount of the water-immiscible organic solvent and an average MW of the HA, is selected such that the formed hydrogel exhibits storage shear modulus (G′) which is higher than that of a hydrogel prepared under the same conditions from the plurality of peptides without a biocompatible polymer. According to some of these embodiments, the G′ value (e.g., when measured as described in the Examples section that follows), is higher by at least 2-folds, or at least 3-folds, or at least 5-folds, or at least 8-fold, or nearly 10-folds.

According to some of any of the embodiments described herein, one or more of a total concentration of plurality of peptides and the biocompatible polymer in the aqueous solution in which it forms, an amount of the water-immiscible organic solvent, a weight ratio between the plurality of peptides and the biocompatible polymer, and an average MW of the HA, is selected such that the formed hydrogel exhibits a storage shear modulus (G′), when measured as described in the Examples section that follows, which is higher than 5 kPa, or higher than 10 kPa, or higher than 20 kPa, preferably higher than 26 kPa, or higher than 30 kPa, more preferably higher than 40 kPa.

According to some of any of the embodiments described herein, a hybrid hydrogel as described herein exhibits a storage shear modulus (G′), when measured as described in the Examples section that follows, which is higher than 20 kPa, or higher than 26 kPa, preferably higher than 30 kPa or higher than 40 kPa, for example, the G′ ranges from 10 to 100, or from 20 to 100, or from 20 to 80, or from 30 to 100, or from 30 to 80, or from 10 to 60, or from 20 to 60, or from 30 to 50, or from 40 to 50, or from 30 to 60, or from 40 to 60, kPa, including any intermediate values and subranges therebetween.

According to some of any of the embodiments described herein, a hybrid hydrogel as described herein exhibits a storage shear modulus (G′), when measured as described in the Examples section that follows, which is higher than that of a hydrogel prepared under the same conditions (e.g., 5 mg/ml) from the plurality of peptides without a biocompatible polymer.

According to some of these embodiments, the G′ value (e.g., when measured as described in the Examples section that follows), is higher by at least 2-folds, or at least 3-folds, or at least 5-folds, or at least 8-fold, or nearly 10-folds.

According to some of any of the embodiments described herein, the composition elicits bone-forming stem cells differentiation.

According to some of any of the embodiments described herein the composition elicits proliferation of bone-forming stem cells and/or osteoblasts differentiated therefrom.

According to some of any of the embodiments described herein the composition elicits migration of bone-forming stem cells and/or osteoblasts differentiated therefrom and/or immune cells that differentiate into M2 macrophages that promote angiogenesis.

According to some of any of the embodiments described herein the composition elicits a vascularized bone regeneration.

According to some of any of the embodiments described herein the composition elicits bone-forming stem cells differentiation.

As used herein “differentiation” refers to the process of commitment to a single cell lineage typically of a mature adult cell. According to a specific embodiment, the differentiation is to a bone cell.

According to a specific embodiment, the composition supports proliferation of bone-forming stem cells and/or osteoblasts differentiated therefrom.

As used herein “proliferation” refers to the process of cell expansion in a given time period as a result of cell growth and division.

According to a specific embodiment, the composition elicits migration of bone-forming stem cells and/or osteoblasts differentiated therefrom and/or immune cells that differentiate into M2 macrophages (se determined by cell surface markers such as CD163 and CD68) that promote angiogenesis.

According to a specific embodiment, the composition elicits a vascularized bone regeneration, thereby generating a high quality tissue.

By governing the macrophage population, the composition is endowed with an immune-modulatory properties.

According to some of any of the embodiments described herein a composition as described herein, which comprises, as a biocompatible polymer hyaluronic acid as described herein in any of the respective embodiments, elicits formation of a bone with higher density than a composition that comprises a polymer other than HA, for example, alginate.

According to some of any of the embodiments described herein, the composition is an immune-modulatory composition.

A composition as described herein can further comprise one or more therapeutically active agents, either incorporated in the hydrogel, or used in combination therewith.

As used herein, the phrase “therapeutically active agent” describes a chemical substance, which exhibits a therapeutic activity when administered to a subject. These include, as non-limiting examples, inhibitors, ligands (e.g., receptor agonists or antagonists), co-factors, anti-inflammatory drugs (steroidal and non-steroidal), anti-psychotic agents, analgesics, anti-thrombogenic agents, anti-platelet agents, anti-coagulants, anti-diabetics, statins, toxins, antimicrobial agents, anti-histamines, metabolites, anti-metabolic agents, vasoactive agents, vasodilator agents, cardiovascular agents, chemotherapeutic agents, antioxidants, phospholipids, anti-proliferative agents and heparins.

As used herein, the phrase “biological substance” refers to a substance that is present in or is derived from a living organism or cell tissue. This phrase also encompasses the organisms, cells and tissues. Representative examples therefore include, without limitation, cells, amino acids, peptides, proteins, oligonucleotides, nucleic acids, genes, hormones, growth factors, enzymes, co-factors, antisenses, antibodies, antigens, vitamins, immunoglobulins, cytokines, prostaglandins, vitamins, toxins and the like, as well as organisms such as bacteria, viruses, fungi and the like.

As used herein, the phrase “diagnostic agent” describes an agent that upon administration exhibits a measurable feature that corresponds to a certain medical condition. These include, for example, labeling compounds or moieties, as is detailed hereinunder.

As used herein, the phrase “labeling compound or moiety” describes a detectable moiety or a probe which can be identified and traced by a detector using known techniques such as spectral measurements (e.g., fluorescence, phosphorescence), electron microscopy, X-ray diffraction and imaging, positron emission tomography (PET), single photon emission computed tomography (SPECT), magnetic resonance imaging (MRI), computed tomography (CT) and the like.

Representative examples of labeling compounds or moieties include, without limitation, chromophores, fluorescent compounds or moieties, phosphorescent compounds or moieties, contrast agents, radioactive agents, magnetic compounds or moieties (e.g., diamagnetic, paramagnetic and ferromagnetic materials), and heavy metal clusters, as is further detailed hereinbelow, as well as any other known detectable moieties.

As used herein, the term “chromophore” refers to a chemical moiety or compound that when attached to a substance renders the latter colored and thus visible when various spectrophotometric measurements are applied.

A heavy metal cluster can be, for example, a cluster of gold atoms used, for example, for labeling in electron microscopy or X-ray imaging techniques.

As used herein, the phrase “fluorescent compound or moiety” refers to a compound or moiety that emits light at a specific wavelength during exposure to radiation from an external source.

As used herein, the phrase “phosphorescent compound or moiety” refers to a compound or moiety that emits light without appreciable heat or external excitation, as occurs for example during the slow oxidation of phosphorous.

As used herein, the phrase “radioactive compound or moiety” encompasses any chemical compound or moiety that includes one or more radioactive isotopes. A radioactive isotope is an element which emits radiation. Examples include α-radiation emitters, β-radiation emitters or γ-radiation emitters.

While a labeling moiety can be attached to the hydrogel, in cases where the one or more of the peptides composing the hydrogel is an end-capping modified peptide, the end-capping moiety can serve as a labeling moiety per se.

Thus, for example, in cases where the Fmoc group described hereinabove is used as the end-capping moiety, the end-capping moiety itself is a fluorescent labeling moiety.

In another example, wherein the Fmoc described hereinabove further includes a radioactive fluoro atom (e.g., ¹⁸F) is used as the end-capping moiety, the end-capping moiety itself is a radioactive labeling moiety.

Other materials which may be incorporated in or on the hybrid hydrogel described herein include, without limitation, conducting materials, semiconducting materials, thermoelectric materials, magnetic materials, light-emitting materials, biominerals, polymers and organic materials.

Each of the agents described herein can be incorporated in or on the hydrogel by means of chemical and/or physical interactions. Thus, for example, compounds or moieties can be attached to the external and/or internal surface of the hydrogel, by interacting with functional groups present within the hydrogel via, e.g., covalent bonds, electrostatic interactions, hydrogen bonding, van der Waals interactions, donor-acceptor interactions, aromatic (e.g., π-π interactions, cation-π interactions and metal-ligand interactions. These interactions lead to the chemical attachment of the material to the fibrous network of the hybrid hydrogel.

As an example, various agents can be attached to the hydrogel via chemical interactions with the side chains, N-terminus or C-terminus of the peptides composing the hydrogel and/or with the end-capping moieties, if present.

Alternatively, various agents can be attached to the hydrogel by physical interactions such as magnetic interactions, surface adsorption, encapsulation, entrapment, entanglement and the likes.

Attachment of the various agents to the hybrid hydrogel can be effected either prior to or subsequent to the hydrogel formation. Thus, for example, an agent or moiety can be attached to one or more of the peptides composing the hydrogel prior to the hydrogel formation, resulting in a hydrogel having the agent attached thereto. Alternatively, an agent or moiety can be attached to surface groups of the hydrogel upon its formation.

Incorporation of the various agents can be effected by forming the hybrid hydrogel in a solution containing the incorporated agent.

Hydrogels entrapping therein a biological or chemical agent can be beneficially utilized for encapsulation and controlled release of the agent.

Hydrogels having a labeling moiety attached thereto or encapsulated therein can be utilized in a variety of applications, including, for example, tracing and tracking the location of the fibrous networks of the present invention in mechanical devices and electronic circuitry; and tracing, tracking and diagnosing concentrations of the hybrid hydrogels of the present invention in a living tissue, cell or host.

According to embodiments of the present invention, a composition as described herein, which comprises a hybrid hydrogel as described herein in any of the respective embodiments and any combination thereof, is a pharmaceutical composition, which can optionally further comprise a pharmaceutically acceptable carrier.

The composition comprising or consisting of a hybrid hydrogel as described herein in any of the respective embodiments can be administered to an organism or otherwise utilized per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the hybrid hydrogel or to a composition comprising same as described herein.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, topical, transmucosal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Optionally and preferably, one may administer the composition or the pharmaceutical composition comprising same in a local rather than systemic manner, for example, via injection of the composition or a pharmaceutical composition comprising same directly onto or into a tissue region of a patient (e.g., a bone tissue), or by implanting the composition in a tissue region of the patient (e.g., a bone defect as described herein).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

According to some embodiments, the pharmaceutical composition as described herein is an injectable composition.

An “injectable composition” as used herein is a composition having a texture and viscosity which permits flow through a suitable delivery device, such as, a surgical needle, or any other suitable applicator, such as a catheter, a cannula, a syringe, a tubular apparatus and so on, as known in the art.

According to some embodiments, the composition or the pharmaceutical composition comprising same is a moldable composition and is formulated for implantation, by means used for local implantation, by injection.

In some of any of these embodiments, the pharmaceutical composition comprises the hybrid hydrogel as described herein, and an aqueous carrier. The carrier can be the aqueous solution used when the hydrogel forms, such that the pharmaceutical composition consists of a composition as described herein in any of the respective embodiments. Alternatively, the aqueous carrier can be added to the hybrid hydrogel after it forms in the aqueous solution and dried (e.g., lyophilized), and is thereafter re-constituted in the aqueous carrier, e.g., sterile, pyrogen-free water based solution.

Compositions of some embodiments of the invention may, if desired, be presented in a pack or dispenser device, such as an FDA approved kit, which may contain one or more unit dosage forms containing the composition as described herein. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

As used herein, the term “subject” includes mammals, preferably human beings at any age which suffer from the pathology. Preferably, this term encompasses individuals who are at risk to develop the pathology.

According to some of any of the embodiments described herein, there is provided a kit that comprises the composition as described herein or a pharmaceutical composition comprising same. The kit may comprise the composition that comprises the hybrid hydrogel, as prepared, or in a dried form thereof (e.g., lyophilized) and optionally can further comprise a pharmaceutically acceptable carrier for reconstituting the hydrogel. Optionally, the kit can comprise the plurality of peptides, the polymer, an aqueous solution, and optionally an organic solvent as described herein in any of respective embodiments, each individually packaged in the kit, along with instructions to mix the components in a weight ratio and a total concentration as described herein in any of the respective embodiments. The kit may further comprises means for administering the composition, for example, means for injecting or implanting the hybrid hydrogel.

According to some of any of the embodiments described herein, the hybrid hydrogel is not processed by electrospinning, and/or is not formed by electrospinning, and/or is not an electrospun hydrogel.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, the term “alkyl” refers to a saturated aliphatic hydrocarbon including straight chain and branched chain groups. Preferably, the alkyl group has 1 to 20 carbon atoms. The alkyl group may be substituted or unsubstituted. When substituted, the substituent group can be, for example, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “cycloalkyl” group refers to an all-carbon monocyclic or fused ring (i.e., rings which share an adjacent pair of carbon atoms) group wherein one or more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

An “alkenyl” group refers to an alkyl group which consists of at least two carbon atoms and at least one carbon-carbon double bond.

An “alkynyl” group refers to an alkyl group which consists of at least two carbon atoms and at least one carbon-carbon triple bond.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroaryl” group refers to a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted. When substituted, the substituent group can be, for example, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. When substituted, the substituted group can be, for example, lone pair electrons, alkyl, trihaloalkyl, alkenyl, alkynyl, cycloalkyl, aryl, heteroaryl, heteroalicyclic, halo, nitro, azo, hydroxy, alkoxy, thiohydroxy, thioalkoxy, cyano, and amine. Representative examples are piperidine, piperazine, tetrahydrofurane, tetrahydropyrane, morpholino and the like.

A “hydroxy” group refers to an —OH group.

A “thio” group (also referred to herein, interchangeably as “thiol” or “thiohydroxy”) refers to a —SH group.

An “azide” group refers to a —N═N≡ group.

An “alkoxy” group refers to both an —O-alkyl and an —O-cycloalkyl group, as defined herein.

An “aryloxy” group refers to both an —O-aryl and an —O-heteroaryl group, as defined herein.

A “thioalkoxy” group refers to both an —S-alkyl group, and an —S-cycloalkyl group, as defined herein.

A “thioaryloxy” group refers to both an —S-aryl and an —S-heteroaryl group, as defined herein.

A “halo” or “halide” group refers to fluorine, chlorine, bromine or iodine.

A “halophenyl” group refers to a phenyl substituted by two, three, four or five halo groups, as defined herein.

A “trihaloalkyl” group refers to an alkyl substituted by three halo groups, as defined herein.

A representative example is trihalomethyl.

An “amino” group refers to an —NR′R″ group where R′ and R″ are hydrogen, alkyl, cycloalkyl or aryl.

A “nitro” group refers to an —NO₂ group.

A “cyano” group refers to a —C≡N group.

For any of the embodiments described herein, the compounds described herein (the peptide and/or the polymer) may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

The compounds described herein (e.g., the peptides and/or polymer as described herein) encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF=CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom. These modifications can occur at any of the bonds along the peptide chain and even at several (2-3) at the same time.

As used herein throughout, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Natural aromatic amino acids, Trp, Tyr and Phe, may be substituted for synthetic unnatural acids such as phenylglycine, TIC, naphthylalanine (Nal), ring-methylated derivatives of Phe, halogenated derivatives of Phe or O-methyl-Tyr, and β amino-acids.

Herein throughout, the phrases “hybrid hydrogel” and “composite hydrogel” are used interchangeably.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Materials and Methods

### Materials:

Lyophilized FmocFF peptide was purchased from Bachem (Budendorf, Switzerland) and high-molecular-weight (3×10⁶ Da) sodium hyaluronate 10 mg/mL in phosphate-buffered saline syringes were purchased from BTG-Ferring (Kiryat Malachy, Israel). Sodium alginate (molecular weight 240 kDa, viscosity 15-25 cP, 1% in H₂O), 3-[4,5-dimethylthiazole-2-yl]-2,5-diphenyltetrazolium bromide (MTT), fluorescein diacetate, propidium iodide, 4-Methylumbelliferyl phosphate (4-MUP), Alizarin red and 9 mm diameter×2 mm depth silicone isolators were purchased from Sigma-Aldrich Inc. (Rehovot, Israel). Calcium Reagent Set was purchased from Pointe Scientific (USA). Dulbecco's Modified Eagle's Medium (DMEM), Minimum Essential Media Alpha (MEMA), fetal bovine serum (FBS), fetal calf serum (FCS), penicillin and streptomycin were purchased from Biological Industries (Beit-Haemek, Israel).

### Structural and Mechanical Characterization of the FmocFF/HA Hydrogel

### FmocFF/HA Composite Hydrogel Preparation

125 mg of HA in phosphate-buffered saline (10 mg/mL) was mixed with 850 μL double distilled water (ddH₂O) using a shaker overnight. FmocFF stock solution was prepared by dissolving the peptide in dimethyl sulfoxide (DMSO) solvent (150 mg peptide in 1 mL of DMSO) by vortexing until a transparent solution was obtained. Peptide stock solution (25 μL) was then added to the mixture of HA in ddH₂O and vortexed. The final concentration of the hydrogel was 5 mg/mL (3.75 mg of FmocFF and 1.25 mg of HA in 1 mL of solution) with 2.5% DMSO.

### Transmitting Electron Microscopy (TEM)

FmocFF/HA composite hydrogel (10 μL) was placed on a 400-mesh copper grid and excess fluid was removed. Negative staining was achieved by the deposition of 10 μL of 2% uranyl acetate in water. After 2 min, excess uranyl acetate was removed. The sample was viewed using a JEM-1400Plus Transmission Electron Microscope (JEM), operating at 80 kV.

### Scanning Electron Microscopy (SEM)

A sample of FmocFF/HA composite hydrogel was placed on a metal stand, freeze-dried and lyophilized, and then coated with a thin gold layer and viewed by SEM (JEOL, JSM-IT100 InTouchScope™) operating at 20 kV.

### Fourier-Transform Infrared (FTIR) Spectroscopy

FTIR spectra were collected four days after hydrogel preparation using a Nicolet Nexus 470 FTIR spectrometer with a DTGS (deuterated triglycine sulfate) detector. Hydrogel samples were placed onto disposable KBr IR sample cards (Sigma-Aldrich, Israel) and vacuum dried. Measurements were performed using 4 cm⁻¹ resolution and by averaging 2000 scans. The absorbance maxima values were determined using the OMNIC analysis program (Nicolet). The obtained transmittance spectra were smoothed by applying the Savitzky-Golay function to eliminate noise and operating the second derivative transformation on the spectra using the Peakfit software version 4.12 (SYSTAT Software Inc., Richmond, CA).

### Rheological Studies

The mechanical properties of the hydrogel were studied with an AR-G2 rheometer (TA Instruments, New Castle, DA, USA) equipped with a Stainless Steel 20 mm diameter parallel-plate geometry. Oscillatory strain (0.01-100%), frequency sweep (0.01-100 Hz), and sheer rate (0.01-100/s) tests were conducted to determine the linear viscoelastic region on 240 μL freshly prepared hydrogel (resulting in a gap size of 0.6 mm) at 37° C. A time-sweep oscillatory test was performed at 5 Hz oscillation and 0.1% strain deformation to determine G′ and G″, namely the storage and loss moduli, respectively. The self-healing behavior of the hydrogel was tested under a cyclic high (100%) and low (0.1%) strain at a constant frequency of 5 Hz.

In Vitro Biological Assays for Assessment of MC3T3-E1 Preosteoblasts Migration within the Hydrogel and Osteogenic Differentiation

All biological assays were performed in triplicate, with five replicates per experiment.

Migration of MC3T3-E1 Preosteoblasts within the Hydrogel

To assess the adherence and migration of cells, the hydrogels were prepared using a 9 mm diameter×2 mm depth silicone mold containing a final volume of 90 μL, placed in a 35 mm glass-bottom dish and repeatedly washed with culture medium for three days, followed by UV sterilization for 30 min. After sterilization, MC3T3-E1 preosteoblasts (20,000 cells per 100 μl) were seeded on the hydrogel. After one day, the cells were stained with fluorescein diacetate (6.6 μg/ml) for 5 min at room temperature to identify cell attachment and migration. The cells were imaged using a Leica SP8×Confocal Microscope.

### Mineralization Assay Using Alizarin Red

Osteogenic differentiation of MC3T3-E1 preosteoblasts on the FmocFF/HA hydrogel was evaluated by the Alizarin red quantification assay and compared to cells seeded on the plate. FmocFF/HA hydrogels were formed in a 24-well plate and repeatedly washed with culture media for three days, followed by UV sterilization for 30 min. A total of 50,000 MC3T3-E1 preosteoblasts per 100 μl were seeded on the prewashed hydrogels and incubated at 37° C. in a humidified atmosphere under 5% CO₂. After two days, the cells were supplemented with osteogenic media containing ascorbic acid and beta-glycerophosphate. The cells were maintained in osteogenic media for 14 days with the media replaced every two days. After 14 days, the cells were stained with the calcium staining dye Alizarin red. Calcium deposits could be visualized by their red color under a light microscope. After washing off the excess dye, optical light microscopy images were acquired. Then, the deposited calcium was extracted by AcOH/MeOH (1:2) buffer. The absorbance at 450 nm was assessed for each well with and without differentiation after subtracting the background binding to the hydrogel itself, and normalized to the cell count. The normalization was performed by counting the number of cells in each well based on cells' nuclei staining with DAPI and fluorescence measurements at 360 nm. The quantification was done using the formula:

$$Normalized absorbance = \frac{Alizarin red absorbance}{DAPI fluorescence} \times 10000$$

### Alkaline Phosphatase (ALP) Activity

To determine the cellular ALP activity, FmocFF/HA hydrogels were formed in a 24-well plate and repeatedly washed with culture medium for three days, followed by UV sterilization for 30 min. A total of 50,000 MC3T3-E1 preosteoblasts per 100 μl were seeded on the prewashed hydrogels. After two days, the cells were supplemented with osteogenic media containing ascorbic acid and beta-glycerophosphate. The cells were maintained in osteogenic media for 14 days with the media replaced every two days. After 14 days, the hydrogels were stained with 100 μl ALP substrate solution containing pNPP and incubated away from light at 37° C. for 30 min. The absorbance at 405 nm was read for each well with and without differentiation and normalized by the number of cells. The normalization was performed by counting the number of cells in each well based on cells' nuclei staining with DAPI and fluorescence measurements at 360 nm. The quantification was done using the formula:

$$Normalized A bsorbance = \frac{ALP absorbance}{DAPI Fluorescence} \times 10000$$

### Calcium Deposition on the FmocFF/HA Hydrogel

FmocFF/HA hydrogels were formed in a 24-well plate and repeatedly washed with culture medium for three days, followed by UV sterilization for 30 min. A total of 50,000 MC3T3-E1 preosteoblasts per 100 μl were seeded on the prewashed hydrogels in osteogenic media containing ascorbic acid and beta-glycerophosphate. Cells were maintained in osteogenic media for 14 days with the media replaced every two days. Following 14 days of induced osteogenic differentiation, the hydrogels were treated with 0.5 N HCl in a cold room (4° C.) on a shaker for 24 h. After 24 h, calcium present in the acidic supernatant was quantified using a commercially available kit (Calcium Reagent Set, Pointe Scientific) following the manufacturer's instructions. Light absorbance of the acidic supernatant solution with the addition of a calcium reagent was read at 570 nm. Calcium content was determined from a standard curve of absorbance against a known concentration of calcium run in parallel with the acidic supernatant solution. Results were normalized over the hydrogel surface area.

### In Vivo Evaluation of the Composite FmocFF/HA Hydrogel's Biocompatibility in a Rat Subcutaneous Model and its Effect on Bone Regeneration in a Calvarial Critical-Sized Defect Model

### Hydrogel Preparation

For the in vivo study, FmocFF/HA hydrogels were prepared using the same protocol and then transferred to one end-closed tubular dialysis membranes 6 mm in diameter (CelluSep® T3 tubing, 12000-14000 MWCO). The other end of the membranes was appropriately closed, and the samples were submerged in 20 mL of DMEM containing 10% fetal calf serum (FCS) and 100 U/mL penicillin, 200 mM L-glutamine in capped 50 mL Falcon® tubes. The medium was replaced eight times over three days before surgery in order to eliminate residual FmocFF monomers and DMSO.

FmocFF/Alg hydrogel was prepared as previously described (Ghosh et al., 2019), transferred into dialysis bags similar to the FmocFF/HA hydrogel and washed with DMEM for three days.

### Biocompatibility Analysis of the Hydrogel in a Rat Subcutaneous Model

The surgical and animal care procedure was reviewed and approved by the committee for the Supervision of Animal Experiments at Tel Aviv University (approval #01-17-078), in compliance with the guidelines for animal experimentation of the National Institutes of Health. FmocFF/HA hydrogels were prepared and soaked with bone marrow harvested from the femor of homologous individuals. A subcutaneous pouch was created by a U-shape incision using a scalpel in the chest of the rats. Simultaneously, bone marrow was harvested from homologous individuals. After euthanization, both ends of the bilateral femora of syngeneic rats were cut, and bone marrow plugs were retrieved. The hydrogel was soaked in the bone marrow in a petri dish. Then, the hydrogel was placed in the subcutaneous pouch, and the incision were sutured. After four weeks, the implant was harvested and placed in ethanol. After decalcification, histologic sections were stained for H&E and imaged using a light microscope (Olympus DP70, Tokyo, Japan).

### Rat Calvarial Bone Defect Model

Surgical and animal care procedures were reviewed and approved by the committee for the Supervision of Animal Experiments at Tel Aviv University (approval #01-18-041), in compliance with the guidelines for animal experimentation of the National Institutes of Health. A minimal number of rats were used, and all efforts were made to minimize potential suffering.

Twenty 8-week old female Sprague-Dawley rats with preoperative weights ranging from 200 to 250 g (Envigo, Israel) were involved. The animals were anesthetized through intraperitoneal injection of Ketamine (100 mg/kg) and Xylazine (5 mg/kg), followed by subcutaneous Enrofloxacin 5% (5 mg/kg) to minimize the risk of infection, and Rimadyl (5 mg/kg) for pain management. Local anesthesia was administered using 2% Lidocaine with 1/100,000 epinephrine. The dorsal part of the cranium was shaved and aseptically prepared with 1% iodine. A U-shaped incision was made, and a full-thickness flap was reflected, exposing the parietal and frontal bones. A critical-size defect of 5 mm diameter was created in the right parietal bone of each rat with a trephine bur under saline irrigation, leaving the left parietal bone intact serving as a control. Care was taken not to damage the dura mater during the surgery. The rats were randomly divided into four groups (n=5). Each defect was filled with either FmocFF/HA hydrogel, FmocFF/Alg hydrogel, or small particles (0.25-1 mm) of deproteinized bovine bone mineral (BBM) (Bio-Oss®, Geistlich Pharma, Wolhusen, Switzerland) as a positive control, or left unfilled as a negative control. Surgical flaps were repositioned, and three single interrupted 5-0 Nylon sutures were placed. The animals were euthanized eight weeks post-surgery by CO₂ asphyxiation, and the calvarias were harvested. The specimens were immediately fixed in 4% paraformaldehyde for 48 h and then placed in 70% ethanol.

### Micro-Computed Tomography (Micro-CT) Analysis

All specimens were scanned using a Scanco micro-CT 50 system (Scanco Medical AG, Switzerland). Scans were performed at an isotropic resolution of 17.2 μm utilizing the following parameters: 34 mm tube, 90 kVp energy, at 200 μA intensity, Al 0.5 mm filter, and with 1000 projections at a 1000 msec integration time. The images were 3D reconstructed in Amira software (v 6.3, www(dot)fei(dot)com) for analysis. A 5 mm diameter circular region of interest (ROI) was analyzed. A corresponding 5 mm circular area in the left parietal bone of each rat served as a control and as a reference for calculations. A color map demonstrating the restored bone thickness in the defect and the corresponding control circular area was drawn. The restored bone volume was calculated as the ratio between the right (experimental) and left (control) ROIs and presented as a percentage. The 5 mm circular defect was then virtually divided into three regions with equal volumes, namely, outer, middle, and inner, and the restored bone volume in each region was calculated and compared between the three regions. The restored bone density in the defined ROIs was calculated from the grayscale values of the micro-CT scans using a calibration phantom provided by the manufacturer (Scanco Medical AG, Switzerland). The ratio between the newly formed bone density to the bone density of the control side was presented as a percentage. All the specimens comprising BBM were segmented using various semi-automatic thresholding tools in Amira software to morphologically separate visible BBM particles from the surrounding bone.

### Histological Preparation

Specimens comprising FmocFF/HA hydrogel, FmocFF/Alg hydrogel, and unfilled defects were fixed with 4% paraformaldehyde and decalcified with 10% EDTA for 21 days. They were then sliced across the center of the defect, embedded in paraffin, and sectioned into 5 μm-thick slices. Bone morphology was visualized by hematoxylin and eosin (H&E) and Masson's trichrome stains. Analysis was performed using a light microscope (Olympus BX-50, Tokyo, Japan) and photomicrographs were acquired using a camera mounted on the microscope (Olympus DP70, Tokyo, Japan).

### Immunohistochemistry for the Assessment of the In Vivo Osteoimmunomodulatory Effect of FmocFF/HA Hydrogel

Eighteen rats were included in this study using the abovementioned protocol. One and 3 weeks after the implantation of either FmocFF/HA hydrogel, BBM, and unfilled defects, 3 rats from each group were euthanized and the calvarias were harvested. The specimens were immediately fixed in 4% paraformaldehyde for 48 h and then placed in 70% ethanol. Decalcification was performed with 10% EDTA for 21 days. The specimens were sliced across the center of the defect, embedded in paraffin, and sectioned into 5 μm-thick slices. The slices were stained with hematoxylin and then labeled with either CD68 or CD163 to determine M1 and M2 macrophage phenotypes, respectively. The slides were scanned in Aperio VERSA Brightfield Digital Scanner (Leica Biosystems, Buffalo Grove, IL 60089, USA) and photomicrographs were taken using Aperio ImageScope—Pathology Slide Viewing Software. M1 and M2 macrophage populations were later quantified and compared between the different mice groups 1 and 3 weeks post implantation using the Image J software. The percentage of M1 and M2 macrophages was quantified in three areas on each slide and averaged, and later compared within the three groups.

Statistical analysis was performed using GraphPad Prism Software (GraphPad Software, Inc., San Diego, CA, USA). Data for each assay were analyzed with two-tailed Student's t-test for comparison between 2 groups or one-way analysis of variance (ANOVA) with Tukey's post-test for multiple comparisons to examine the effect of the hydrogel and the controls on the parameters investigated. For all tests, statistical significance was set at p=0.05.

### Example 2

### Characterization of the FmocFF/HA Hydrogel

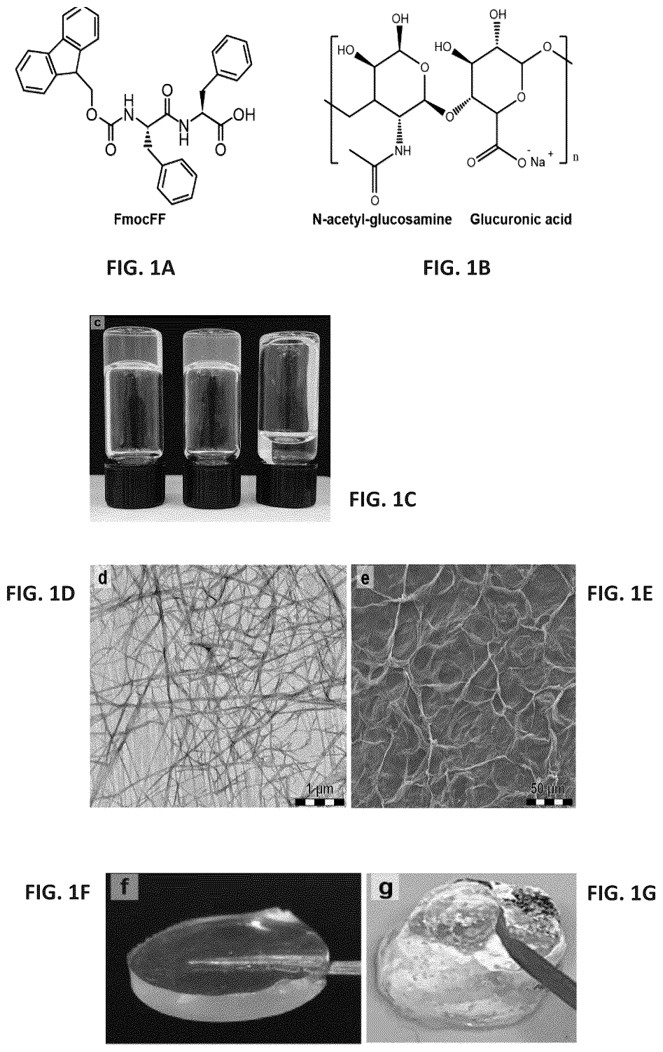

The FmocFF/HA composite hydrogel was formed by incorporating FmocFF (FIG. 1A) into HA (FIG. 1B) to a final concentration of 5 mg/Ml. This gave rise to a self-supported, homogenous hydrogel formed within a few minutes (FIG. 1C). TEM and SEM analyses demonstrate the nanofibrillar architecture of the composite hydrogel (FIGS. 1D-E). The composite hydrogel is moldable and easy to handle. It can be customized to different configurations (FIG. 1F) or applied through a syringe (FIG. 1G).

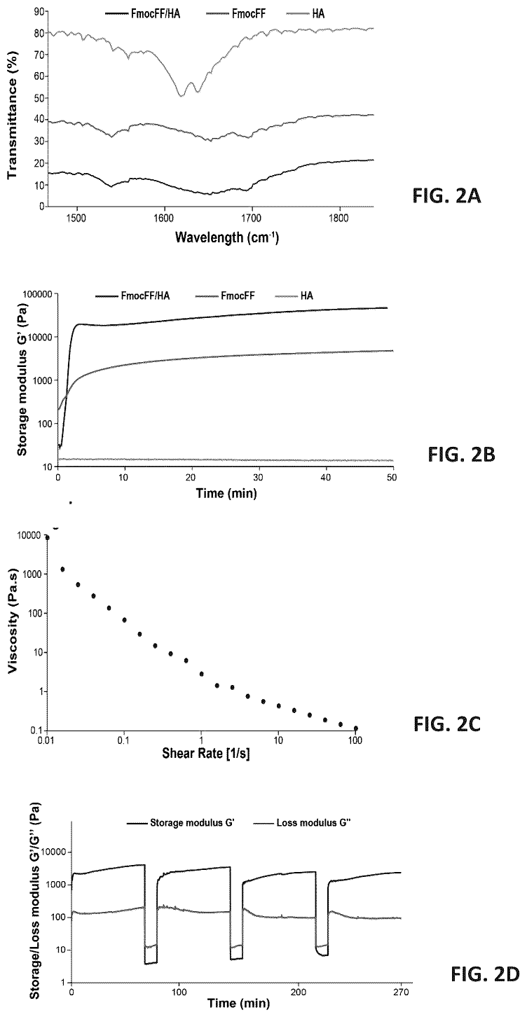

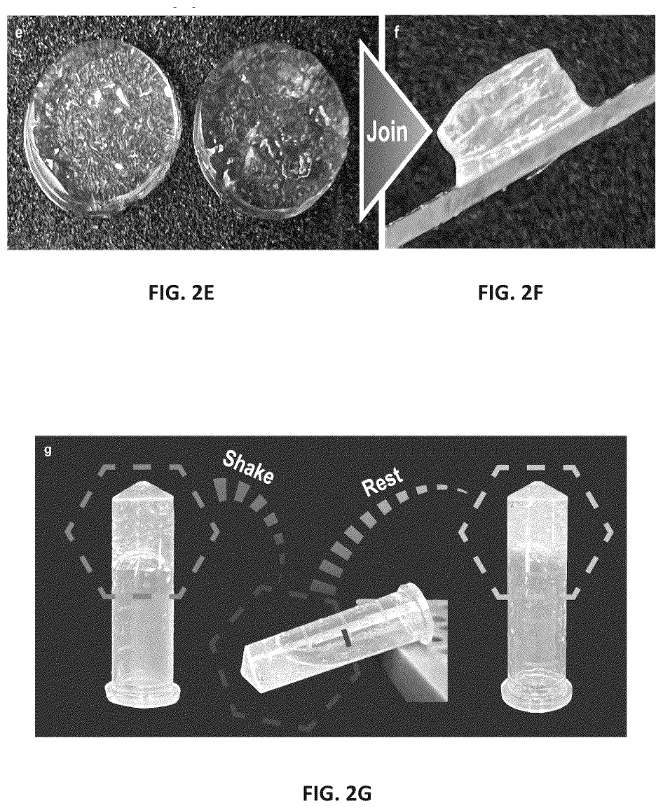

FTIR spectra were measured for the FmocFF/HA composite hydrogel and the individual components (FIG. 2A). The distinct peaks observed at 1,647 cm⁻¹ and at 1,694 cm⁻¹ are associated with amide I C═O stretching vibration and are directly related to the backbone conformation (Di Foggia et al., 2011). These peaks suggest the presence of a carbamate moiety and indicate that the composite hydrogel is rich in β-sheets. HA showed a peak at 1,415 cm⁻¹ that corresponds to the presence of a C—O group with C═O combination, and additional peak at 1,620 cm⁻¹ that indicates amide II stretching (Di Foggia et al., 2011, Kong and Yu, 2007). These peaks are not visible in the composite hydrogel.

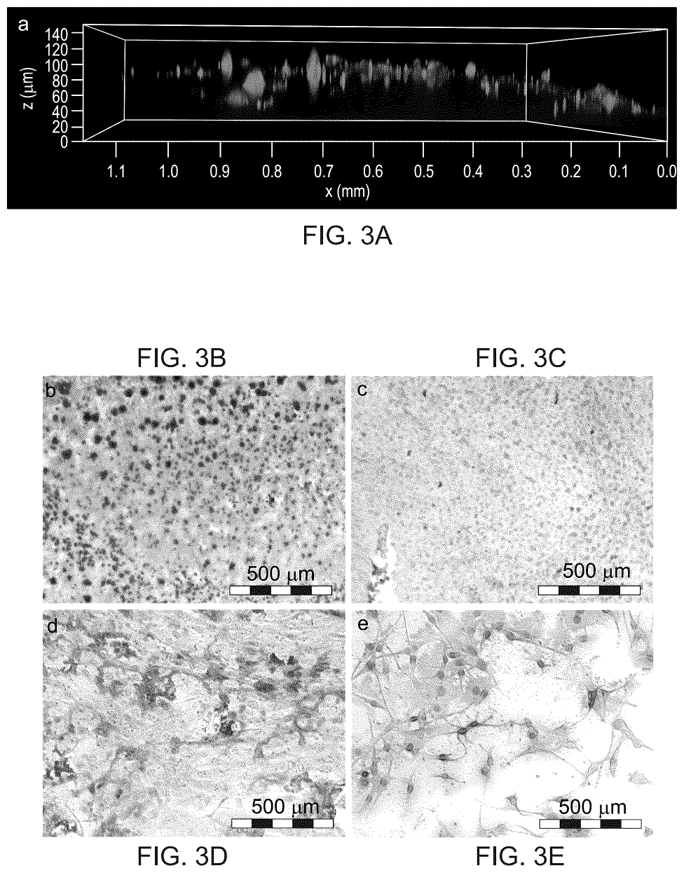

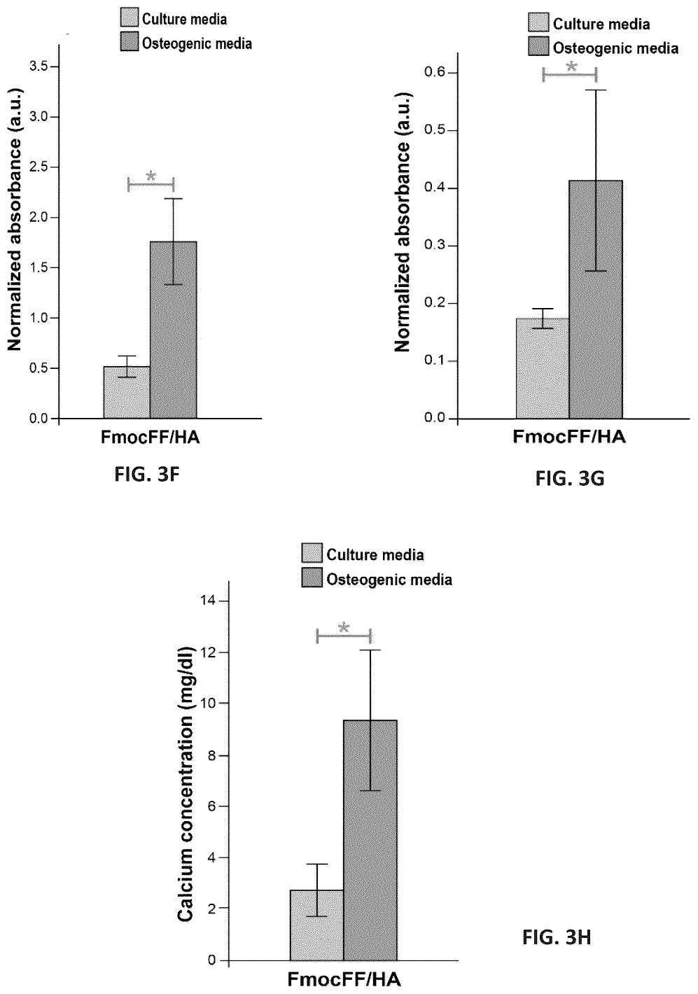

To assess the stiffness of the FmocFF/HA composite hydrogel, the present inventors first measured the storage modulus and the loss modulus of the composite hydrogel at dynamic strain sweep (at 5 Hz frequency). To study the effect of oscillatory strain, the hydrogel was subjected to 0.01-100% strain sweep at a constant frequency, resulting in a broad linear viscoelastic region of up to 5% strain (not shown). Frequency sweep experiments at a constant strain using a frequency range of 0.1-100 Hz also showed a broad linear viscoelastic region (not shown). The linear viscoelastic region was determined based on both the dynamic strain sweep and frequency sweep tests and conducted a time sweep measurement at a fixed strain of 0.1% and frequency of 5 Hz. The FmocFF/HA composite hydrogel reached a G′ value of 46,425 Pa, which was 9.8-fold higher than the pure FmocFF with a G of 4,708 Pa (FIG. 2B). HA alone had a very low G′ value of 13 Pa (FIG. 2B). FIG. 2C shows the viscosity variation of the FmocFF/HA composite hydrogel with respect to shear rate (0.01-100/s). The composite hydrogel showed shear-induced breakage, supporting a shear-thinning behavior of the gel. At low shear rate, the high viscosity value of the composite hydrogel might have stemmed from the entangled networks of polymer chains and fibrils (Chakraborty et al., 2012). As the shear rate increased, the viscosity value decreased. This is due to the network rearrangements in the microstructure in the plane of the applied shear. The entanglements were disrupted by the imposed deformation, resulting in the breakage of the fibril network and depletion of the gel. To establish the hydrogel's self-healing nature, an alternate step strain experiment was conducted. The hydrogel was subjected to seven cycles of time sweep experiments with low (0.1%) and high (100%) strain values (FIG. 2D). At 100% strain, the hydrogels were converted to a sol state, as evident from the modulus values (G′<G″). When the strain was reduced to 0.1%, the hydrogel recovered (G′>G″). This characteristic was consistent over multiple cycles, illustrating the reproducibility of the self-healing nature. The self-healing property of the hydrogel was also observed by the joining of three separate disc hydrogels (FIGS. 2E-F). Moreover, the recovery of the hydrogel was achieved after applying mechanical force (FIG. 3G).

### Example 3

### In Vitro Assessment of the Biocompatibility of the Hydrogel and its Effect on Osteodifferentiation of MC3T3-E1 Preosteoblasts

The present inventors assessed the migration and osteoinductive potential of the FmocFF/HA composite hydrogel by using it as a substrate for the growth of MC3T3-E1 preosteoblasts. Cell migration could be observed already one day post-seeding using confocal microscopy (FIG. 3A). Moreover, the hydrogel efficiently induced the differentiation and mineralization of MC3T3-E1 preosteoblasts. Matrix mineralization was quantified by Alizarin red staining calculated 14 days after cell seeding, with and without osteogenic differentiation. Staining was normalized to the number of cells. FIGS. 3B-C show that when seeded on the FmocFF/HA hydrogel, the intensity of Alizarin red staining was higher for cells grown in osteogenic media (FIG. 3B) compared to cells treated grown in culture media (FIG. 3C). The staining intensity is also higher compared to cell seeded on the plate with and without differentiation media (FIGS. 3D-E). As shown in FIG. 3F, the quantified intensity of Alizarin red staining was higher in differentiated cells than in cells without differentiation media (p=0.0126). This result was confirmed by the normalized quantification of Alkaline phosphatase (ALP) activity the cells grown on the hydrogel and of calcium content in the supernatant of the hydrogel (FIG. 3G). Fourteen days after osteogenic differentiation, an increase in ALP activity was observed compared to cells seeded on the hydrogels and treated with culture media (p=0.0143, FIG. 3G). In addition, higher amounts of calcium deposits were found in the supernatant of the hydrogel when compared to undifferentiated cells grown for 14 days in regular culture media (p=0.0211, FIG. 3H) or osteogenic media (p=0.0328). Finally, higher calcium deposition was observed in the supernatant of the hydrogel with osteoinductive agents than without osteogenic media (p=0.0177, FIG. 3H).

### Example 4

### In Vivo Assessment of Bone Regeneration in Critical-Sized Defects with FmocFF/HA Composite Hydrogel

### Micro-CT Analysis of Critical-Sized Bone Defects In Vivo

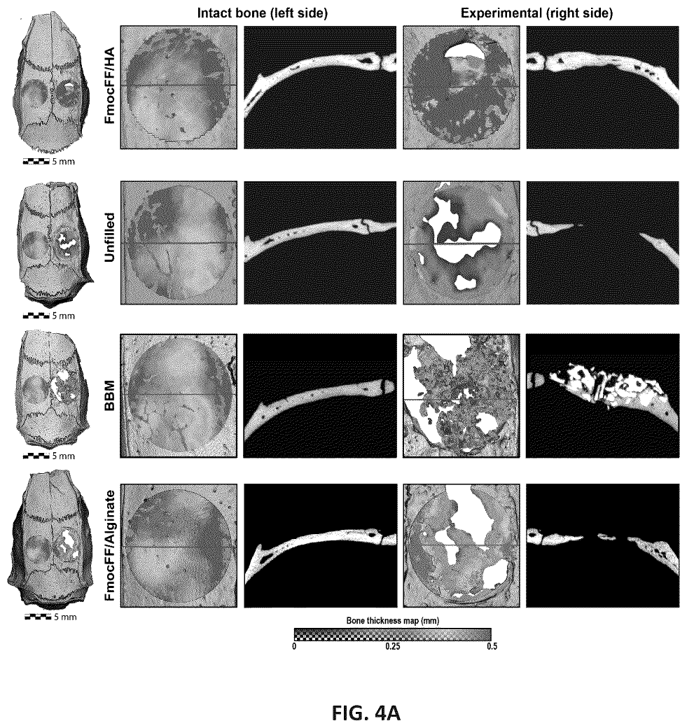

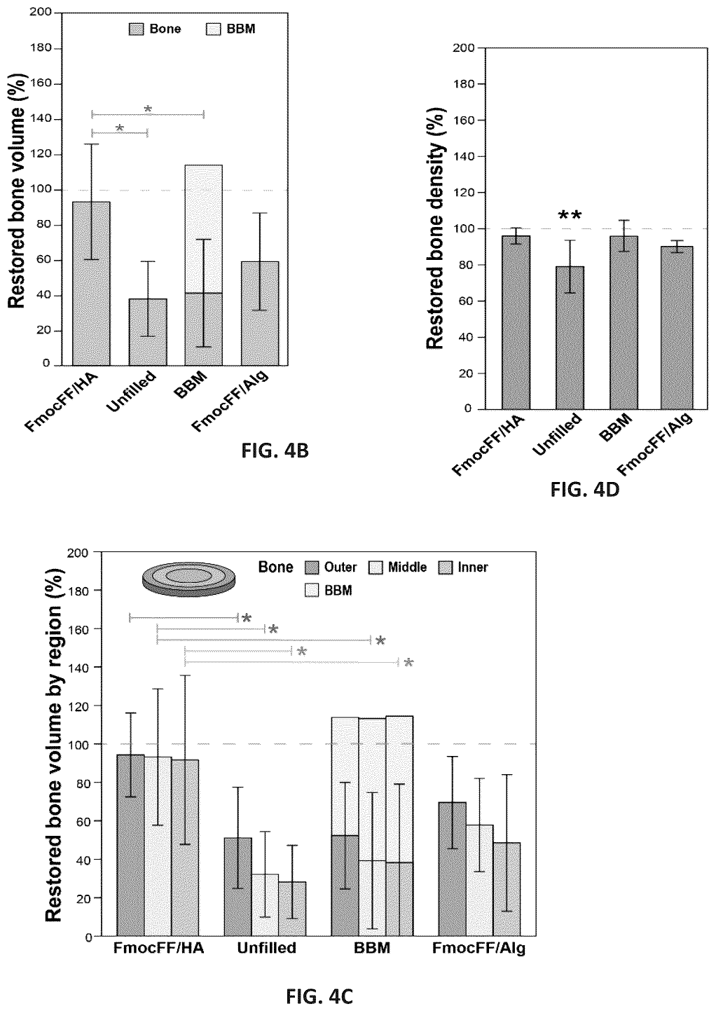

In order to assess the potential of the FmocFF/HA hydrogel to serve as a scaffold for bone regeneration, the present inventors investigated its ability to induce bone formation in a rat calvarial critical-sized defect model. The hydrogel's stiffness (G′) was confirmed and found to be 48,780 Pa. A critical-size defect 5 mm in diameter was created in the right parietal bone of each rat. Each defect was filled with either FmocFF/HA hydrogel, small particles of deproteinized BBM, FmocFF/Alg hydrogel (positive controls) or left unfilled (a negative control). All animals survived the implantation surgery and exhibited uneventful postoperative healing. Eight weeks after implantation, hydrogel remnants were not observed in the treated sites. Newly formed bone was observed in all groups (FIG. 4A). Coronal cross-sections of the defects in the four groups revealed that unfilled defects contained newly formed bone only at the defect margins, while, in the defects filled with FmocFF/HA, BBM, or FmocFF/Alg, the newly formed bone bridged the defect. Notably, the newly formed mineralized tissue in the FmocFF/HA-filled defect was indistinguishable from the surrounding native bone. In the FmocFF/Alg treatment, the newly formed bone was homologous to the native bone, however, its thickness decreased towards the center of the defect with some unfilled areas. In the BBM-filled defects, bony islands could be observed in direct contact with the graft particles and particularly adjacent to the dural side. Residual BBM particles were observed in the defect area, with some particles extending outside of the defect area close to the periosteal side.

The top view color map demonstrates a decrease in bone thickness from the defect margins inward in the unfilled defect group as well as in the FmocFF/Alg hydrogel group. In contrast, in the FmocFF/HA hydrogel group, the thickness of the newly formed bone was homogeneous throughout the defect, with a similar or even higher thickness than of the untreated control side. The thickness of the newly formed bone in the BBM-filled defects was relatively low and inconsistent across the region (FIG. 4A).

The mean restored bone volume was calculated and compared between the four groups (FIG. 4B). In the FmocFF/HA hydrogel group, the restored bone volume was 92.95±32.77%. Significantly lower values of 37.89±21.27% (p=0.0256), 41.20±30.46% (p=0.0355), and 59.06±27.70% were observed in the unfilled defect, BBM, and FmocFF/Alg groups, respectively (FIG. 4B). The majority of the defect (72.52±34.99%) in the BBM group was composed of residual graft particles. To investigate further, the restored bone volume in three different regions of the defect, namely, outer, middle, and inner areas, were compared (FIG. 4C). In the unfilled defects, there was a trend to a decrease in the mean bone volume when moving from the edges of the defect towards the inner section, with values of 51.11±23.59% at the periphery but 32.13±19.94% in the mid-section, and 28.15±17.06% in the inner. A similar trend was observed in the BBM group, where the mean bone volumes decreased from 51.95±24.77% at the edge, through 38.77±31.71% in the mid-section, to 31.41±30.33%, in the inner, and in the FmocFF/Alg hydrogel where the bone volumes decreased from 69.97±24.06% at the edge, through 58.22±24.20% in the mid-section, to 49±35.60% in the inner. Notably, in the FmocFF/HA hydrogel treatments, bone volumes were seen throughout the repaired defect, with similar values across the three regions: 94.23±19.55%, 93.16±31.79%, and 91.64±39.37%, in the outer, middle, and inner regions, respectively. The differences in mean bone volume between the FmocFF/HA and the unfilled defect groups were statistically significant in all three regions (p=0.0489, p=0.0257, and p=0.0298 for the outer, middle, and inner regions, respectively). A statistically significant difference in mean bone volume was observed between the FmocFF/HA and the BBM groups in the middle and inner regions (p=0.0463 and p=0.0390, respectively).

FIG. 4D demonstrates the newly formed bone density, calculated relative to the intact contralateral bone. In both the FmocFF/HA and the BBM groups, the new bone density was almost similar to that of the original bone, with values of 95.94±3.86% (p=0.7217) and 95.98±7.72% (p>0.9999), respectively. A lower bone density value of 79.03±13.05% was observed in the unfilled defect group, which was significantly different (p=0.0083) from the original bone.

### Histological Evaluation

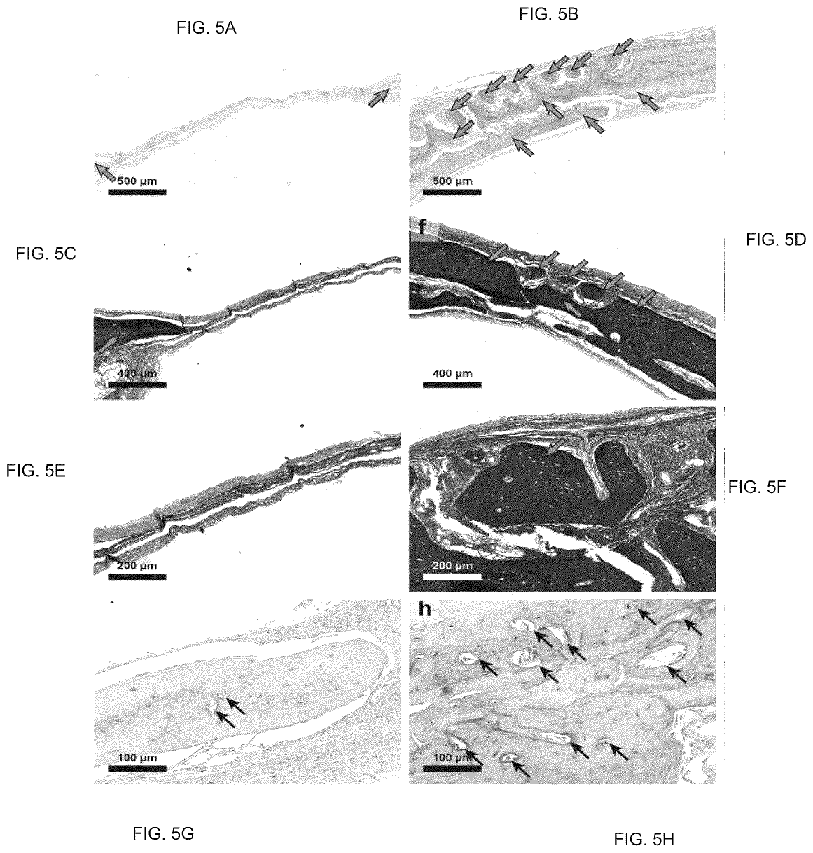

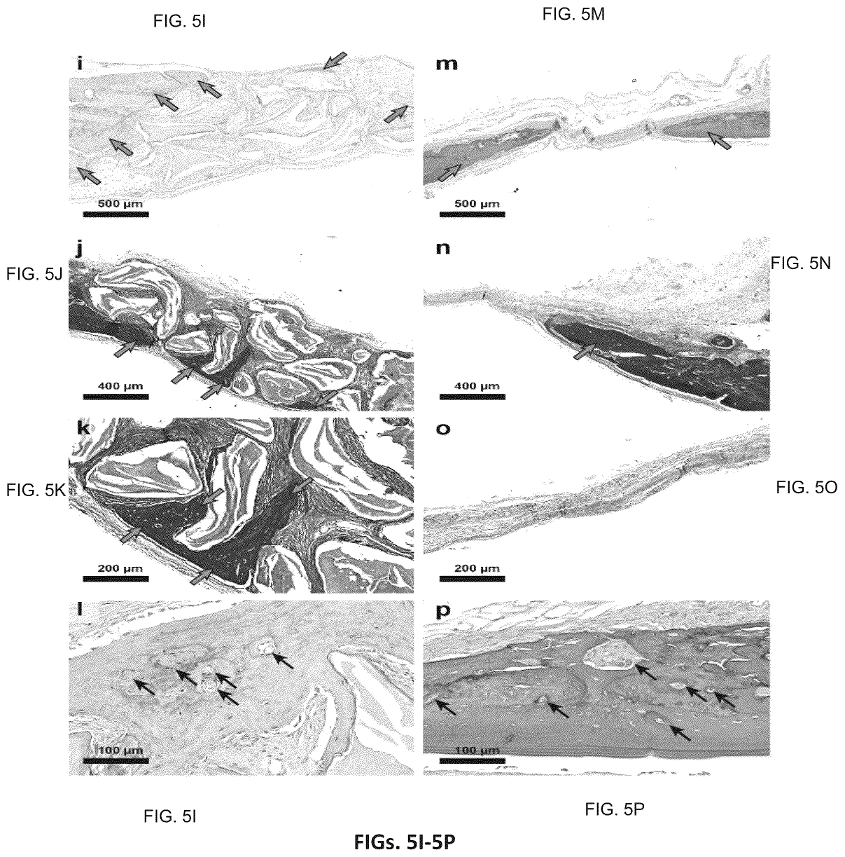

In order to complement the Micro-CT evaluation, the present inventors performed histological analysis. Following eight weeks, the unfilled defects contained newly mineralized tissue, including new blood vessels, extending from the defect margins (FIGS. 5A-D), while the central region of the defects was filled with fibrous connective tissue comprising fibroblasts and blood vessels. In contrast, the FmocFF/HA hydrogel-filled defects contained higher amounts of new bone formation that not only originated from the defect margins but was also evident in the central portion of the defects (FIG. 5E). At higher magnification, islands of newly formed bone that do not start at the margins of the defect were evident (FIGS. 5F-G). Both osteocytes and new blood vessels entrapped in these bone islands indicated the viability of the newly formed bone (FIGS. 5G-H). In the defects filled with BBM, several particles surrounded by newly formed bone were evident, while most of the particles were surrounded by connective tissue (FIGS. 5I-K). The newly formed bone was mostly continuous with the defect margins, however, the bone formed around the BBM particles included entrapped osteocytes and blood vessels, indicating the new bone's viability (FIG. 5L). In the FmocFF/Alg hydrogel-filled defect, similar to the unfilled defects, new bone arising from the defect edges with new blood vessels could be observed (FIGS. 5M-P), as in the negative control.

### Osteoimmunomodulation of the FmocFF/HA Hydrogel

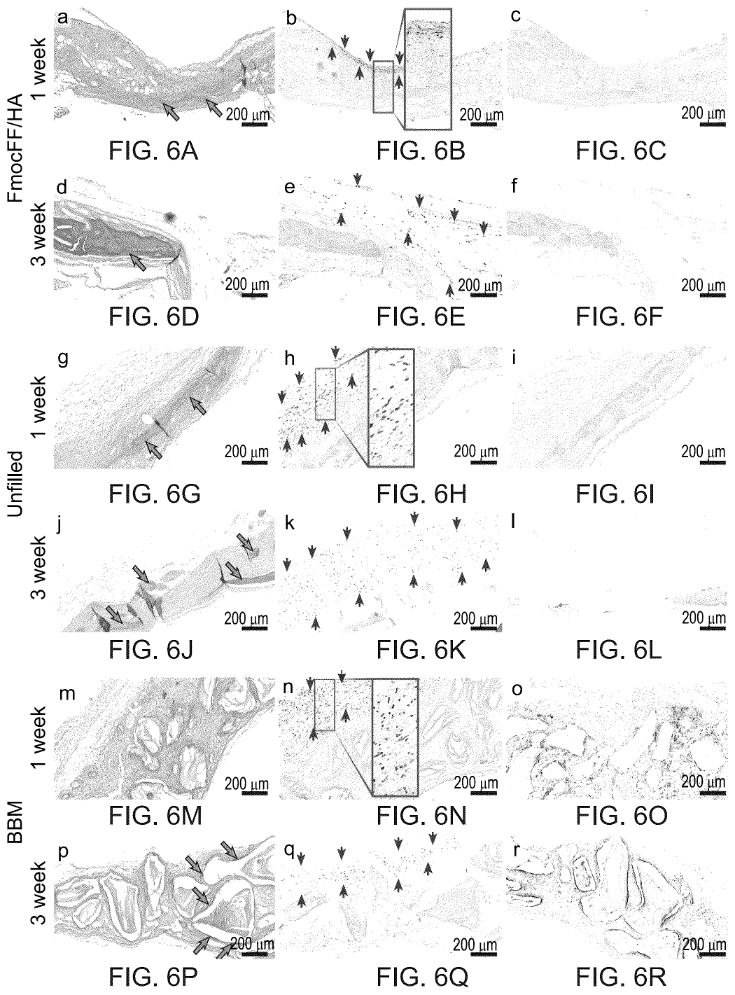

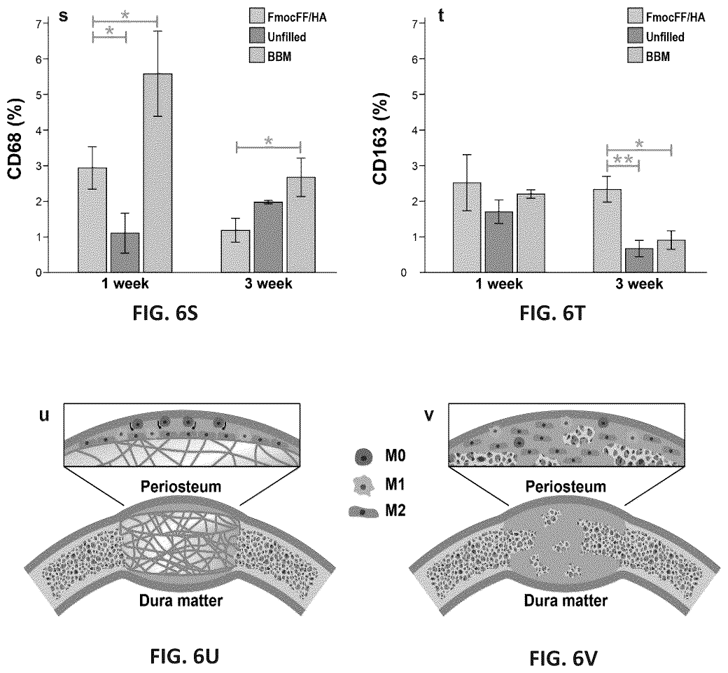

laving demonstrated that the Fmoc FF/HA hydrogel resulted in complete restoration of the calvarial critical sized-defect, the present inventors sought to determine whether macrophage polarization may play a role in this process compared to unfilled defects and to defects filled with BBM. CD68 and CD163 immunohistochemical labeling was used to visualize M1 (pro-inflammatory) and M2 (pro-regenerative) macrophages, respectively, in the calvar al defect (Tournier et al., 2021). Baseline levels of M2 and M1 macrophages at 1 and 3 weeks after implantation were observed in the intact left parietal side (not shown). One-week post-implantation, a low amount of M2 and M1 macrophages can be observed under the periosteum (not shown, respectively). At three weeks after implantation, slightly higher amounts of M2 and M1 macrophages can be detected (not shown, respectively), In the FmocFF/HA hydrogel group, one week after implantation, a beginning of bone formation could be seen in the middle of the defect (FIG. 6A). Elongated M2 macrophages were detected at the hydrogel-periosteum interface (FIG. 6B), while M1 macrophages were hard to discern (FIG. 6C). At three weeks of regeneration, bigger bone islands could be observed (FIG. 6D). M2 macrophages were observed above and under the newly formed bone adjacent to the periosteum and the dura mater, respectively, and between the bone islands throughout the regenerating tissue (FIG. 6E). At this time point as well, M1 macrophages were hard to discern (FIG. 6F). In the unfilled defects, M2 macrophages could be observed both at one week and three weeks, while M1 population was hardly observed (FIG. 6G-L). In the BBM group one week after implantation, both M1 and M2 macrophages could be observed. The M2 population was located under the periosteum on top of the BBM particles, while the M1 population was seen also under the periosteum but mainly surrounding the BBM particles. Following three weeks, both M1 and M2 macrophages were seen between the BBM particles (FIGS. 6M-R). Interestingly, quantification of M1 and M2 macrophages shows that in the FmocFF/HA hydrogel group, at 1 week M1>M2 while at 3 weeks M2>M1 (FIG. 6S-T). A reduction in M1 was observed from 1 week to 3 weeks. In the unfilled defect, M2>M1 at 1 week, however, at 3 weeks M11>M2. In the B13M group, M1>M2 at both time points. Notably, after three weeks, the FmocFF/HA hydrogel group demonstrated low presence of M1 cells and the highest presence of M2 cell compared to BBM or unfilled defect groups (p=0.0298 and p=0.0526 for M1, respectively and p=0.0181 and p=0.0025 for M2, respectively). These results may suggest the effect of the FmocFF/HA hydrogel on macrophage polarization into M2 macrophages, which may play a key role in bone regeneration in vivo (FIGS. 6U-V).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

ADDIN EN.REFLIST Adams, D. J., Butler, M. F., Frith, W. J., Kirkland, M., Mullen, L. & Sanderson, P. (2009) A new method for maintaining homogeneity during liquid-hydrogel transitions using low molecular weight hydrogelators. Soft Matter. 5, 1856-1862.
Adams, D. J., Mullen, L. M., Berta, M., Chen, L. & Frith, W. J. (2010) Relationship between molecular structure, gelation behaviour and gel properties of Fmoc-dipeptides. Soft Matter. 6, 1971-1980.
Alakpa, Enateri V., Jayawarna, V., Lampel, A., Burgess, Karl V., West, Christopher C., Bakker, Sanne C. J., Roy, S., Javid, N., Fleming, S., Lamprou, Dimitris A., Yang, J., Miller, A., Urquhart, Andrew J., Frederix, Pim W. J. M., Hunt, Neil T., Péault, B., Ulijn, Rein V. & Dalby, Matthew J. (2016) Tunable Supramolecular Hydrogels for Selection of Lineage-Guiding Metabolites in Stem Cell Cultures. Chem 1, 298-319.
Aviv, M., Halperin-Sternfeld, M., Grigoriants, I., Buzhansky, L., Mironi-Harpaz, I., Seliktar, D., Einav, S., Nevo, Z. & Adler-Abramovich, L. (2018) Improving the Mechanical Rigidity of Hyaluronic Acid by Integration of a Supramolecular Peptide Matrix. ACS Appl. Mater. Interfaces 10, 41883-41891.
Bai, X., Gao, M., Syed, S., Zhuang, J., Xu, X. & Zhang, X. Q. (2018) Bioactive hydrogels for bone regeneration. Bioact. Mater. 3, 401-417.
Beniash, E., Hartgerink, J. D., Storrie, H., Stendahl, J. C. & Stupp, S. I. (2005) Self-assembling peptide amphiphile nanofiber matrices for cell entrapment. Acta Biomater. 1, 387-397.
Boerckel, J. D., Uhrig, B. A., Willett, N. J., Huebsch, N. & Guldberg, R. E. (2011) Mechanical regulation of vascular growth and tissue regeneration in vivo. Proc. Natl. Acad. Sci. U.S.A. 108, E674-E680.
Brito, A., Abul-Haija, Y. M., da Costa, D. S., Novoa-Carballal, R., Reis, R. L., Ulijn, R. V., Pires, R. A. & Pashkuleva, I. (2019) Minimalistic supramolecular proteoglycan mimics by co-assembly of aromatic peptide and carbohydrate amphiphiles. Chem. Sci. 10, 2385-2390.
Brown, B. N., Ratner, B. D., Goodman, S. B., Amar, S. & Badylak, S. F. (2012) Macrophage polarization: an opportunity for improved outcomes in biomaterials and regenerative medicine. Biomaterials 33, 3792-3802.
Celik, E., Bayram, C., Akcapinar, R., Turk, M. & Denkbas, E. B. (2016) The effect of calcium chloride concentration on alginate/Fmoc-diphenylalanine hydrogel networks. Mater. Sci. Eng. C. Mater. Biol. Appl. 66, 221-229.
Chakraborty, P., Roy, B., Bairi, P. & Nandi, A. K. (2012) Improved mechanical and photophysical properties of chitosan incorporated folic acid gel possessing the characteristics of dye and metal ion absorption. J. Mater. Chem. 22, 20291-20298.
Cheng, W., Ding, Z., Zheng, X., Lu, Q., Kong, X., Zhou, X., Lu, G. & Kaplan, D. L. (2020) Injectable hydrogel systems with multiple biophysical and biochemical cues for bone regeneration. Biomater. Sci. 8, 2537-2548.
Chircov, C., Grumezescu, A. M. & Bejenaru, L. E. (2018) Hyaluronic acid-based scaffolds for tissue engineering. Rom. J. Morphol. Embryol. 59, 71-76.
Csoka, A. B., Frost, G. I. & Stem, R. (2001) The six hyaluronidase-like genes in the human and mouse genomes. Matrix Biol. 20, 499-508.
Debnath, S., Roy, S., Abul-Haija, Y. M., Frederix, P., Ramalhete, S. M., Hirst, A. R., Javid, N., Hunt, N. T., Kelly, S. M., Angulo, J., Khimyak, Y. Z. & Ulijn, R. V. (2019) Tunable Supramolecular Gel Properties by Varying Thermal History. Chemistry 25, 7881-7887.
Deidda, G., Jonnalagadda, S. V. R., Spies, J. W., Ranella, A., Mossou, E., Forsyth, V. T., Mitchell, E. P., Bowler, M. W., Tamamis, P. & Mitraki, A. (2017) Self-Assembled Amyloid Peptides with Arg-Gly-Asp (RGD) Motifs As Scaffolds for Tissue Engineering. ACS Biomater. Sci. Eng. 3, 1404-1416.
Derkus, B., Okesola, B. O., Barrett, D. W., D'Este, M., Chowdhury, T. T., Eglin, D. & Mata, A. (2020) Multicomponent hydrogels for the formation of vascularized bone-like constructs in vitro. Acta Biomater. 109, 82-94.
Di Foggia, M., Taddei, P., Torreggiani, A., Dettin, M. & Tinti, A. (2011) Self-Assembling Peptides for Biomedical Applications: IR and Raman Spectroscopies for the Study of Secondary Structure. Proteomics Res. J. 2, 231.
Diaferia, C., Gianolio, E. & Accardo, A. (2019a) Peptide-based building blocks as structural elements for supramolecular Gd-containing MRI contrast agents. J. Pept. Sci. 25, e3157.
Diaferia, C., Morelli, G. & Accardo, A. (2019b) Fmoc-diphenylalanine as a suitable building block for the preparation of hybrid materials and their potential applications. J. Mater. Chem. B. 7, 5142-5155.
Diaferia, C., Netti, F., Ghosh, M., Sibillano, T., Giannini, C., Morelli, G., Adler-Abramovich, L. & Accardo, A. (2020) Bi-functional peptide-based 3D hydrogel-scaffolds. Soft Matter. 16, 7006-7017.
Dou, X. Q. & Feng, C. L. (2017) Amino Acids and Peptide-Based Supramolecular Hydrogels for Three-Dimensional Cell Culture. Adv. Mater. 29, 1604062.
Draper, E. R. & Adams, D. J. (2019) Controlling the Assembly and Properties of Low-Molecular-Weight Hydrogelators. Langmuir 35, 6506-6521.
Duchamp de Lageneste, O., Julien, A., Abou-Khalil, R., Frangi, G., Carvalho, C., Cagnard, N., Cordier, C., Conway, S. J. & Colnot, C. (2018) Periosteum contains skeletal stem cells with high bone regenerative potential controlled by Periostin. Nat. Commun. 9, 773.
Eliezer, M., Imber, J. C., Sculean, A., Pandis, N. & Teich, S. (2019) Hyaluronic acid as adjunctive to non-surgical and surgical periodontal therapy: a systematic review and meta-analysis. Clin. Oral Investig. 23, 3423-3435.
Engler, A. J., Sen, S., Sweeney, H. L. & Discher, D. E. (2006) Matrix elasticity directs stem cell lineage specification. Cell 126, 677-689.
Fichman, G. & Gazit, E. (2014) Self-assembly of short peptides to form hydrogels: design of building blocks, physical properties and technological applications. Acta Biomater. 10, 1671-1682.
Franz, S., Rammelt, S., Scharnweber, D. & Simon, J. C. (2011) Immune responses to implants—a review of the implications for the design of immunomodulatory biomaterials. Biomaterials 32, 6692-6709.
Gallo, E., Diaferia, C., Gregorio, E. D., Morelli, G., Gianolio, E. & Accardo, A. (2020) Peptide-Based Soft Hydrogels Modified with Gadolinium Complexes as MRI Contrast Agents. Pharmaceuticals 13.
Ghosh, M., Halperin-Sternfeld, M., Grigoriants, I., Lee, J., Nam, K. T. & Adler-Abramovich, L. (2017) Arginine-Presenting Peptide Hydrogels Decorated with Hydroxyapatite as Biomimetic Scaffolds for Bone Regeneration. Biomacromolecules 18, 3541-3550.
Ghosh, M., Halperin-Sternfeld, M., Grinberg, I. & Adler-Abramovich, L. (2019) Injectable Alginate-Peptide Composite Hydrogel as a Scaffold for Bone Tissue Regeneration. Nanomaterials 9, 497.
Gong, X., Branford-White, C., Tao, L., Li, S., Quan, J., Nie, H. & Zhu, L. (2016) Preparation and characterization of a novel sodium alginate incorporated self-assembled Fmoc-FF composite hydrogel. Mater. Sci. Eng. C. Mater. Biol. Appl. 58, 478-486.
Halperin-Sternfeld, M., Ghosh, M., Sevostianov, R., Grigoriants, I. & Adler-Abramovich, L. (2017) Molecular co-assembly as a strategy for synergistic improvement of the mechanical properties of hydrogels. Chem. Commun. 53, 9586-9589.
Halperin-Sternfeld, M., Netanel Liberman, G., Kannan, R., Netti, F., Ma, P. X., Arad, S. M. & Adler-Abramovich, L. (2022) Thixotropic Red Microalgae Sulfated Polysaccharide-Peptide Composite Hydrogels as Scaffolds for Tissue Engineering. Biomedicines 10, 1388.
Hao, S., Meng, J., Zhang, Y., Liu, J., Nie, X., Wu, F., Yang, Y., Wang, C., Gu, N. & Xu, H. (2017) Macrophage phenotypic mechanomodulation of enhancing bone regeneration by superparamagnetic scaffold upon magnetization. Biomaterials 140, 16-25.
Hartgerink, J. D., Beniash, E. & Stupp, S. I. (2002) Peptide-amphiphile nanofibers: a versatile scaffold for the preparation of self-assembling materials. Proc. Natl. Acad. Sci. U.S.A 99, 5133-5138.
Hemshekhar, M., Thushara, R. M., Chandranayaka, S., Sherman, L. S., Kemparaju, K. & Girish, K. S. (2016) Emerging roles of hyaluronic acid bioscaffolds in tissue engineering and regenerative medicine. Int. J. Biol. Macromol. 86, 917-928.
Heslop, J. A., Hammond, T. G., Santeramo, I., Tort Piella, A., Hopp, I., Zhou, J., Baty, R., Graziano, E. I., Proto Marco, B., Caron, A., Skold, P., Andrews, P. W., Baxter, M. A., Hay, D. C., Hamdam, J., Sharpe, M. E., Patel, S., Jones, D. R., Reinhardt, J., Danen, E. H., Ben-David, U., Stacey, G., Bjorquist, P., Piner, J., Mills, J., Rowe, C., Pellegrini, G., Sethu, S., Antoine, D. J., Cross, M. J., Murray, P., Williams, D. P., Kitteringham, N. R., Goldring, C. E. & Park, B. K. (2015) Concise review: workshop review: understanding and assessing the risks of stem cell-based therapies. Stem Cells Transl. Med. 4, 389-400.
Holzwarth, J. M. & Ma, P. X. (2011) Biomimetic nanofibrous scaffolds for bone tissue engineering. Biomaterials 32, 9622-9629.
Huang, R., Qi, W., Feng, L., Su, R. & He, Z. (2011) Self-assembling peptide-polysaccharide hybrid hydrogel as a potential carrier for drug delivery. Soft Matter. 7, 6222-6230.
Ischakov, R., Adler-Abramovich, L., Buzhansky, L., Shekhter, T. & Gazit, E. (2013) Peptide-based hydrogel nanoparticles as effective drug delivery agents. Bioorg. Med. Chem. 21, 3517-3522.
Jayawarna, V., Ali, M., Jowitt, T. A., Miller, A. F., Saiani, A., Gough, J. E. & Ulijn, R. V. (2006a) Nanostructured Hydrogels for Three-Dimensional Cell Culture Through Self-Assembly of Fluorenylmethoxycarbonyl-Dipeptides. Adv. Mater. 18, 611-614.
Jayawarna, V., Ali, M., Jowitt, T. A., Miller, A. F., Saiani, A., Gough, J. E. & Ulijn, R. V. (2006b) Nanostructured hydrogels for three-dimensional cell culture through self-assembly of fluorenylmethoxycarbonyl-dipeptides. Adv. Mater. 18, 611-614.
Jayawarna, V., Richardson, S. M., Hirst, A. R., Hodson, N. W., Saiani, A., Gough, J. E. & Ulijn, R. V. (2009) Introducing chemical functionality in Fmoc-peptide gels for cell culture. Acta Biomater. 5, 934-943.
Khadeja, L., Grigoriants, I., Halperin-Sternfeld, M., Yona, A. & Adler-Abramovich, L. (2019) Sonochemical Functionalization of Cotton and Non-Woven Fabrics with Bio-Inspired Self-Assembled Nanostructures. Isr. J. Chem. 60, 1190-1196.
Kisiday, J., Jin, M., Kurz, B., Hung, H., Semino, C., Zhang, S. & Grodzinsky, A. J. (2002) Self-assembling peptide hydrogel fosters chondrocyte extracellular matrix production and cell division: implications for cartilage tissue repair. Proc. Natl. Acad. Sci. U.S.A 99, 9996-10001.
Kobayashi, T., Chanmee, T. & Itano, N. (2020) Hyaluronan: Metabolism and Function. Biomolecules 10, 1525.
Kong, J. & Yu, S. (2007) Fourier Transform Infrared Spectroscopic Analysis of Protein Secondary Structures. Acta Biochim. Biophys. Sin. 39, 549-559.
Kretsinger, J. K., Haines, L. A., Ozbas, B., Pochan, D. J. & Schneider, J. P. (2005) Cytocompatibility of self-assembled beta-hairpin peptide hydrogel surfaces. Biomaterials 26, 5177-5186.
Kubler, N. & Urist, M. R. (1990) Bone morphogenetic protein-mediated interaction of periosteum and diaphysis. Citric acid and other factors influencing the generation of parosteal bone. Clin. Orthop. Relat. Res., 279-294.
Liu, M., Tolg, C. & Turley, E. (2019) Dissecting the Dual Nature of Hyaluronan in the Tumor Microenvironment. Front. Immunol. 10.
Loffler, J., Sass, F. A., Filter, S., Rose, A., Ellinghaus, A., Duda, G. N. & Dienelt, A. (2019) Compromised Bone Healing in Aged Rats Is Associated With Impaired M2 Macrophage Function. Front. Immunol. 10.
Mahler, A., Reches, M., Rechter, M., Cohen, S. & Gazit, E. (2006) Rigid, self-assembled hydrogel composed of a modified aromatic dipeptide. Adv. Mater. 18, 1365-1370.
McCloskey, A. P., Draper, E. R., Gilmore, B. F. & Laverty, G. (2017) Ultrashort self-assembling Fmoc-peptide gelators for anti-infective biomaterial applications. J Pept Sci 23, 131-140.
McWhorter, F. Y., Wang, T., Nguyen, P., Chung, T. & Liu, W. F. (2013) Modulation of macrophage phenotype by cell shape. Proc. Natl. Acad. Sci. U.S.A. 110, 17253-17258.
Monzon, M. E., Fregien, N., Schmid, N., Falcon, N. S., Campos, M., Casalino-Matsuda, S. M. & Forteza, R. M. (2010) Reactive oxygen species and hyaluronidase 2 regulate airway epithelial hyaluronan fragmentation. J. Biol. Chem. 285, 26126-26134.
Mosser, D. M. & Edwards, J. P. (2008) Exploring the full spectrum of macrophage activation. Nat. Rev. Immunol. 8, 958-969.
Murray, P. J. & Wynn, T. A. (2011) Protective and pathogenic functions of macrophage subsets. Nat. Rev. Immunol. 11, 723-737.
Nadernezhad, A., Forster, L., Netti, F., Adler-Abramovich, L., TeBmar, J. & Groll, J. (2020) Rheological analysis of the interplay between the molecular weight and concentration of hyaluronic acid in formulations of supramolecular HA/FmocFF hybrid hydrogels. Polym. J. 52, 1007-1012.
Netti, F., Aviv, M., Dan, Y., Rudnick-Glick, S., Halperin-Sternfeld, M. & Adler-Abramovich, L. (2022) Stabilizing gelatin-based bioinks under physiological conditions by incorporation of ethylene-glycol-conjugated Fmoc-FF peptides. Nanoscale 14, 8525-8533.
Ni, M., Zhuo, S., Iliescu, C., So, P. T. C., Mehta, J. S., Yu, H. & Hauser, C. A. E. (2019) Self-assembling amyloid-like peptides as exogenous second harmonic probes for bioimaging applications. J. Biophotonics 12, e201900065.
Park, J. W., Jang, J. H., Bae, S. R., An, C. H. & Suh, J. Y. (2009) Bone formation with various bone graft substitutes in critical-sized rat calvarial defect. Clin. Oral Implants Res. 20, 372-378.
Pek, Y. S., Wan, A. C. & Ying, J. Y. (2010) The effect of matrix stiffness on mesenchymal stem cell differentiation in a 3D thixotropic gel. Biomaterials 31, 385-391.
Pelham, R. J., Jr. & Wang, Y. (1997) Cell locomotion and focal adhesions are regulated by substrate flexibility. Proc. Natl. Acad. Sci. U.S.A 94, 13661-13665.
Qiu, P., Li, M., Chen, K., Fang, B., Chen, P., Tang, Z., Lin, X. & Fan, S. (2020) Periosteal matrix-derived hydrogel promotes bone repair through an early immune regulation coupled with enhanced angio- and osteogenesis. Biomaterials 227, 119552.
Rachmiel, D., Anconina, I., Rudnick-Glick, S., Halperin-Sternfeld, M., Adler-Abramovich, L. & Sitt, A. (2021) Hyaluronic Acid and a Short Peptide Improve the Performance of a PCL Electrospun Fibrous Scaffold Designed for Bone Tissue Engineering Applications. Int. J. Mol. Sci. 22.
Raeburn, J., Pont, G., Chen, L., Cesbron, Y., Lévy, R. & Adams, D. J. (2012) Fmoc-diphenylalanine hydrogels: understanding the variability in reported mechanical properties. Soft Matter. 8, 1168-1174.
Rodon Fores, J., Bigo-Simon, A., Wagner, D., Payrastre, M., Damestoy, C., Blandin, L., Boulmedais, F., Kelber, J., Schmutz, M., Rabineau, M., Criado-Gonzalez, M., Schaaf, P. & Jierry, L. (2021) Localized Enzyme-Assisted Self-Assembly in the Presence of Hyaluronic Acid for Hybrid Supramolecular Hydrogel Coating. Polymers 13.
Ruppert, S. M., Hawn, T. R., Arrigoni, A., Wight, T. N. & Bollyky, P. L. (2014) Tissue integrity signals communicated by high-molecular weight hyaluronan and the resolution of inflammation. Immunol. res. 58, 186-192.
Seib, F. P., Prewitz, M., Werner, C. & Bornhauser, M. (2009) Matrix elasticity regulates the secretory profile of human bone marrow-derived multipotent mesenchymal stromal cells (MSCs). Biochem. Biophys. Res. Commun. 389, 663-667.
Shao, A., Ling, Y., Xu, L., Liu, S., Fan, C., Wang, Z., Xu, B. & Wang, C. (2018) Xenogeneic bone matrix immune risk assessment using GGTA1 knockout mice. Artif Cells Nanomed. Biotechnol. 46, S359-S369.
Silber, J. S., Anderson, D. G., Daffner, S. D., Brislin, B. T., Leland, J. M., Hilibrand, A. S., Vaccaro, A. R. & Albert, T. J. (2003) Donor site morbidity after anterior iliac crest bone harvest for single-level anterior cervical discectomy and fusion. Spine 28, 134-139.
Smith, A. M., Williams, R. J., Tang, C., Coppo, P., Collins, R. F., Turner, M. L., Saiani, A. & Ulijn, R. V. (2008) Fmoc-Diphenylalanine Self Assembles to a Hydrogel via a Novel Architecture Based on π-π Interlocked β-Sheets. Adv. Mater. 20, 37-41.
Sohn, J.-Y., Park, J.-C., Um, Y.-J., Jung, U.-W., Kim, C.-S., Cho, K.-S. & Choi, S.-H. (2010) Spontaneous healing capacity of rabbit cranial defects of various sizes. J. Periodontal Implant Sci. 40, 180-187.
Stevens, M. M. (2008) Biomaterials for bone tissue engineering. Mater. Today 11, 18-25.
Sudo, H., Kodama, H. A., Amagai, Y., Yamamoto, S. & Kasai, S. (1983) In vitro differentiation and calcification in a new clonal osteogenic cell line derived from newborn mouse calvaria. J. Cell Biol. 96, 191-198.
Tikhonova, T., Rovnyagina, N., Arnon, Z., Yakimov, B., Efremov, Y., Cohen-Gerassi, D., Kosheleva, N., Drachev, V., Svistunov, A., Timashev, P., Adler-Abramovich, L., Shirshin, E. & Halperin-Sternfeld, M. (2021) Mechanical Enhancement and Kinetics Regulation of Fmoc-Diphenylalanine Hydrogels by Thioflavin T. Angew. Chem. Int. Ed. Engl.
Tiwari, S. & Bahadur, P. (2019) Modified hyaluronic acid based materials for biomedical applications. Int. J. Biol. Macromol. 121, 556-571.
Tournier, P., Guicheux, J., Paré, A., Maltezeanu, A., Blondy, T., Veziers, J., Vignes, C., André, M., Lesoeur, J., Barbeito, A., Bardonnet, R., Blanquart, C., Corre, P., Geoffroy, V., Weiss, P. & Gaudin, A. (2021) A partially demineralized allogeneic bone graft: in vitro osteogenic potential and preclinical evaluation in two different intramembranous bone healing models. Sci. Rep. 11, 4907.
Turley, E. A., Noble, P. W. & Bourguignon, L. Y. (2002) Signaling properties of hyaluronan receptors. J. Biol. Chem. 277, 4589-4592.
Vacanti, J. P. & Langer, R. (1999) Tissue engineering: the design and fabrication of living replacement devices for surgical reconstruction and transplantation. Lancet 354 Suppl 1, S132-SI34.
Vegners, R., Shestakova, I., Kalvinsh, I., Ezzell, R. M. & Janmey, P. A. (1995) Use of a gel-forming dipeptide derivative as a carrier for antigen presentation. J. Pept. Sci. 1, 371-378.
Wang, L., Gong, C., Yuan, X. & Wei, G. (2019) Controlling the Self-Assembly of Biomolecules into Functional Nanomaterials through Internal Interactions and External Stimulations: A Review. Nanomaterials 9, 285.
Wang, Y., Zhang, Z., Xu, L., Li, X. & Chen, H. (2013) Hydrogels of halogenated Fmoc-short peptides for potential application in tissue engineering. Colloids Surf B. Biointerfaces 104, 163-168.
Wende, F. J., Gohil, S., Mojarradi, H., Gerfaud, T., Nord, L. I., Karlsson, A., Boiteau, J.-G., Kenne, A. H. & Sandström, C. (2016) Determination of substitution positions in hyaluronic acid hydrogels using NMR and MS based methods. Carbohydr. Polym. 136, 1348-1357.
Witzler, M., Buchner, D., Shoushrah, S. H., Babczyk, P., Baranova, J., Witzleben, S., Tobiasch, E. & Schulze, M. (2019) Polysaccharide-Based Systems for Targeted Stem Cell Differentiation and Bone Regeneration. Biomolecules 9.
Woo, K. M., Chen, V. J. & Ma, P. X. (2003) Nano-fibrous scaffolding architecture selectively enhances protein adsorption contributing to cell attachment. J. Biomed. Mater. Res. A. 67, 531-537.
Woo, K. M., Jun, J. H., Chen, V. J., Seo, J., Baek, J. H., Ryoo, H. M., Kim, G. S., Somerman, M. J. & Ma, P. X. (2007) Nano-fibrous scaffolding promotes osteoblast differentiation and biomineralization. Biomaterials 28, 335-343.
Yadav, N., Chauhan, M. K. & Chauhan, V. S. (2020) Short to ultrashort peptide-based hydrogels as a platform for biomedical applications. Biomater. Sci. 8, 84-100.
Yamamoto, H., Tobisawa, Y., Inubushi, T., Irie, F., Ohyama, C. & Yamaguchi, Y. (2017) A mammalian homolog of the zebrafish transmembrane protein 2 (TMEM2) is the long-sought-after cell-surface hyaluronidase. J. Biol. Chem. 292, 7304-7313.
Yang, H. C., Park, H. C., Quan, H. & Kim, Y. (2018) Immunomodulation of Biomaterials by Controlling Macrophage Polarization. Adv. Exp. Med. Biol. 1064, 197-206.
Yoshida, H., Nagaoka, A., Kusaka-Kikushima, A., Tobiishi, M., Kawabata, K., Sayo, T., Sakai, S., Sugiyama, Y., Enomoto, H. & Okada, Y. (2013) KIAA1199, a deafness gene of unknown function, is a new hyaluronan binding protein involved in hyaluronan depolymerization. PNAS 110, 5612-5617.
Zhai, P., Peng, X., Li, B., Liu, Y., Sun, H. & Li, X. (2020) The application of hyaluronic acid in bone regeneration. Int. J. Biol. Macromol. 151, 1224-1239.
Zhang, H., Yang, L., Yang, X. G., Wang, F., Feng, J. T., Hua, K. C., Li, Q. & Hu, Y. C. (2019) Demineralized Bone Matrix Carriers and their Clinical Applications: An Overview. Orthop. Surg. 11, 725-737.
Zhang, R. & Ma, P. X. (2000) Synthetic nano-fibrillar extracellular matrices with predesigned macroporous architectures. J. Biomed. Mater. Res. A. 52, 430-438.
Zhang, S. (2003) Fabrication of novel biomaterials through molecular self-assembly. Nat. Biotechnol. 21, 1171-1178.
Zhang, S., Marini, D. M., Hwang, W. & Santoso, S. (2002) Design of nanostructured biological materials through self-assembly of peptides and proteins. Curr. Opin. Chem. Biol. 6, 865-871.
Zhang, Y., Gu, H., Yang, Z. & Xu, B. (2003) Supramolecular hydrogels respond to ligand-receptor interaction. J. Am. Chem. Soc. 125, 13680-13681.
Zhao, X.-B., Chen, Y.-P., Tan, M., Zhao, L., Zhai, Y.-Y., Sun, Y.-L., Gong, Y., Feng, X.-Q., Du, J. & Fan, Y.-B. (2021) Extracellular Matrix Stiffness Regulates DNA Methylation by PKCα-Dependent Nuclear Transport of DNMT3L. Adv. Healthc. Mater. 10, 2100821.
Zhou, M., Smith, A. M., Das, A. K., Hodson, N. W., Collins, R. F., Ulijn, R. V. & Gough, J. E. (2009) Self-assembled peptide-based hydrogels as scaffolds for anchorage-dependent cells. Biomaterials 30, 2523-2530.
Zuk, P. A., Zhu, M., Mizuno, H., Huang, J., Futrell, J. W., Katz, A. J., Benhaim, P., Lorenz, H. P. & Hedrick, M. H. (2001) Multilineage cells from human adipose tissue: implications for cell-based therapies. Tissue Eng. 7, 211-228.

What is claimed is:

1. A method of inducing bone regeneration, the method comprising contacting a periosteum of a bone with a composition comprising a hybrid hydrogel comprising a biocompatible polymer and a plurality of self-assembled peptides, thereby inducing bone regeneration, wherein:
each peptide in said plurality of peptides is a dipeptide which comprises at least one aromatic amino acid residue; and
said biocompatible polymer is or comprises hyaluronic acid having an average molecular weight of at least 100, at least 500, or at least 1,000 kDa.

2. The method of claim 1, wherein said contacting is performed ex-vivo.

3. The method of claim 1, wherein said contacting is performed in-vivo in a subject in need thereof.

4. The method of claim 1, wherein said composition further comprises an agent selected from the group consisting of an osteo-inductive agent, an anti-inflammatory agent and antibiotics.

5. The method of claim 4, wherein said osteo-inductive drug comprises a proteinaceous agent.

6. The method of claim 5, wherein said proteinaceous agent is selected from the group consisting of include morphogenic protein (BMP), transforming growth factor-β protein (TGF-β), insulin type growth factor (IGF), and fibroblast growth factor (FGF), specifically, basic fibroblast growth factor (bFGF) or Vascular Endotherial Growth Factor (VEGF).

7. The method of claim 3, wherein said subject suffers from a medical condition associated with a bone fracture and/or bone defect.

8. The method of claim 7, wherein said medical condition is a fracture.

9. The method of claim 7, wherein said subject is in need of a dental implant or has been implanted with said dental implant and said composition preserves and/or regenerates an alveolar bone surrounding said dental implant.

10. The method of claim 1, wherein said bone is a cranial bone.

11. The method of claim 7, wherein said medical condition is resorption of alveolar bone optionally said resorption of alveolar bone is associated with a missing tooth.

12. The method of claim 7, wherein said medical condition is a bone growth disorder.

13. The method of claim 1, wherein the hybrid hydrogel forms upon contacting said plurality of peptides and said biocompatible polymer in an aqueous solution.

14. The method of claim 13, where a total concentration of said peptides and said biocompatible polymer in said aqueous solution is 5 mg/mg.

15. The method of claim 1, wherein each of said dipeptides consists of aromatic amino acid residues.

16. The method of claim 1, wherein each dipeptide in said plurality of dipeptides is a homodipeptide.

17. The method of claim 1, wherein each of said dipeptides is phenylalanine-phenylalanine dipeptide (Phe-Phe).

18. The method of claim 1, wherein each dipeptide in said plurality of peptides is an end-capping modified peptide optionally wherein each of said end-capping modified dipeptides comprises an aromatic end-capping moiety optionally said aromatic end capping moiety is 9-fluorenylmethyloxycarbonyl (Fmoc).

19. The method of claim 1, wherein a weight ratio of said dipeptides and said polymer in said aqueous solution ranges from 10:1 to 1:10, or from 5:1 to 1:5, or from 5:1 to 1:1, or from 5:1 to 2:1, or is about 3:1.

20. The method of claim 1, wherein the hybrid hydrogel is characterized by a storage modulus G′ higher than 1,000 kPa, or higher than 2,000 kPa, or higher than 3,000 kPa, or higher than 4,000 kPa, at 5 Hz frequency and at 25° C.
